SEQUENCE LISTING (24 sequences)

SEQ ID NO: 1 - protein, 248 aa, Homo sapiens
MWLCPLALNLILMAASGAACEVKDVCVGSPGIPGTPGSHGLPGRDGRDGVKGDPGPPGPMGPPGETPCPPGNNGLPGAPGVPGERGEKGEAGERGPPGLPAHLDEELQATLHDFRHQILQTRGALSLQGSIMTVGEKVFSSNGQSITFDAIQEACARAGGRIAVPRNPEENEAIASFVKKYNTYAYVGLTEGPSPGDFRYSDGTPVNYTNWYRGEPAGRGKEQCVEMYTDGQWNDRNCLYSRLTICEF

SEQ ID NO: 2 - protein, 10 aa, Homo sapiens
PAGRGKEQCV

SEQ ID NO: 3 - protein, 10 aa, Homo sapiens
EMYTDGQWND

SEQ ID NO: 4 - protein, 10 aa, Homo sapiens
KEQCVEMYTD

SEQ ID NO: 5 - protein, 10 aa, Homo sapiens
PAGRGKEKCV

SEQ ID NO: 6 - protein, 10 aa, Homo sapiens
KEKCVEMYTD

SEQ ID NO: 7 - protein, 20 aa, Homo sapiens
PAGRGKEKCVEMYTDGQWND

SEQ ID NO: 8 - protein, 20 aa, Homo sapiens
PAGRGKEQCVEMYTDGQWND

SEQ ID NO: 9 - protein, 6 aa, Homo sapiens
KEQCVE

SEQ ID NO: 10 - protein, 7 aa, synthetic construct
  REGION 1..7: note = Artificial Peptide Sequence
  VARIANT 7: note = X is any natural or non-natural amino acid
  REPEAT 7
KEQCVEX

SEQ ID NO: 11 - protein, 10 aa, Homo sapiens
  MOD_RES 1: note = ACETYLATION
  MOD_RES 1: note = AMIDATION
KEQCVEMYTD

SEQ ID NO: 12 - protein, 12 aa, Homo sapiens
  MOD_RES 1: note = ACETYLATION
  MOD_RES 12: note = AMIDATION
WGKEQCVEMYTD

SEQ ID NO: 13 - protein, 10 aa, synthetic construct
  REGION 1..10: note = Artificial Peptide Sequence
  REPEAT 1..10
  MOD_RES 1: note = ACETYLATION
  MOD_RES 10: note = AMIDATION
KEQCVEMYTD

SEQ ID NO: 14 - protein, 10 aa, synthetic construct
  REGION 1..10: note = Artificial Peptide Sequence
  MOD_RES 1: note = ACETYLATION
  SITE 10: note = MISC_FEATURE - acid
KEQCVEMYTD

SEQ ID NO: 15 - protein, 10 aa, synthetic construct
  REGION 1..10: note = Artificial Peptide Sequence
  SITE 1: note = MISC_FEATURE - Histidine
  SITE 10: note = MISC_FEATURE - acid
KEQCVEMYTD

SEQ ID NO: 16 - protein, 10 aa, synthetic construct
  REGION 1..10: note = Artificial Peptide Sequence
  MOD_RES 1: note = ACETYLATION
  MOD_RES 7: note = Nle
  MOD_RES 10: note = AMIDATION
KEQCVELYTD

SEQ ID NO: 17 - protein, 12 aa, synthetic construct
  REGION 1..12: note = Artificial Peptide Sequence
  MOD_RES 1: note = ACETYLATION
  MOD_RES 9: note = Nle
  MOD_RES 12: note = AMIDATION
WGKEQCVELYTD

SEQ ID NO: 18 - protein, 12 aa, synthetic construct
  REGION 1..12: note = Artificial Peptide Sequence.
  MOD_RES 1: note = ACETYLATION
  MOD_RES 7: note = Nle
  MOD_RES 12: note = AMIDATION
RGKEQCVELYTD

SEQ ID NO: 19 - protein, 12 aa, synthetic construct
  REGION 1..12: note = Artificial Peptide Sequence
  MOD_RES 1: note = D-tryptophan
  MOD_RES 1: note = ACETYLATION
  MOD_RES 9: note = Nle
  MOD_RES 12: note = AMIDATION
WGKEQCVELYTD

SEQ ID NO: 20 - protein, 8 aa, synthetic construct
  REGION 1..8: note = Artificial Peptide Sequence
  VARIANT 1: note = X is any natural or non-natural amino acid
  VARIANT 8: note = X is any natural or non-natural amino acid
XKEQCVEX

SEQ ID NO: 21 - protein, 12 aa, synthetic construct
  MOD_RES 9: note = Nle
  MOD_RES 12: note = Pego3 modification
  MOD_RES 12: note = AMIDATION
  MOD_RES 1: note = ACETYLATION
WGKEQCVELYTD

SEQ ID NO: 22 - protein, 9 aa, synthetic construct
  MOD_RES 9: note = Nle
  MOD_RES 9: note = Pego3 modification
  MOD_RES 9: note = AMIDATION
  MOD_RES 1: note = ACETYLATION
WGKEQCVEL

SEQ ID NO: 23 - protein, 12 aa, synthetic construct
  MOD_RES 9: note = Nle
  MOD_RES 12: note = Pego3 Modification
  MOD_RES 1: note = ACETYLATION
  MOD_RES 12: note = LIPIDATION
WGKEQCVELYTD

SEQ ID NO: 24 - protein, 9 aa, synthetic construct
  MOD_RES 9: note = Nle
  MOD_RES 9: note = Pego3 Modification
  MOD_RES 9: note = LIPIDATION
  MOD_RES 1: note = ACETYLATION
WGKEQCVEL

(19) United States
(12) Patent Application Publication
(10) Pub. No.: US 2024/0269242 A1
(43) Pub. Date: Aug. 15, 2024

(54) COMPOSITIONS AND METHODS FOR TREATING AND PREVENTING LUNG DISEASE

(71) Applicants: ARIZONA BOARD OF REGENTS ON BEHALF OF THE UNIVERSITY OF ARIZONA, Tucson, AZ (US); RAESEDO, LLC, Tucson, AZ (US)

(72) Inventors: Julie Ledford, Tucson, AZ (US); Monica Kraft, New York, NY (US); Josef Vagner, Tucson, AZ (US)

(21) Appl. No.: 18/586,133

(22) Filed: Feb. 23, 2024

Related U.S. Application Data
(63) Continuation-in-part of application No. PCT/US22/75356, filed on Aug. 23, 2022.
Application No. PCT/US22/75356 is a continuation-in-part of application No. 17/409,642, filed on Aug. 23, 2021.
(63) Continuation-in-part of application No. 17/409,642, filed on Aug. 23, 2021.
Application No. 17/409,642 is a continuation-in-part of application No. 16/093,063, filed on Oct. 11, 2018, now Pat. No. 11,110,152.
Application No. 17/409,642 is a continuation-in-part of application No. PCT/US2021/026411, filed on Apr. 8, 2021.
(60) Provisional application No. 62/320,950, filed on Apr. 11, 2016.
(60) Provisional application No. 63/006,831, filed on Apr. 8, 2020.

(51) Int. Cl. A61K 38/17; A61P 11/06; A61P 31/14; C07K 14/785

(52) U.S. Cl. CPC A61K 38/395; A61P 11/06; A61P 31/14; C07K 14/785

(57) ABSTRACT

Provided herein are compositions and methods for treating and preventing lung disease. In particular, provided herein are SP-A peptides and uses thereof in the treatment and prevention of lung disease (e.g., asthma or COPD). The compositions may comprise peptides having an amino acid sequence of Ac-WGKEQCVE(Nle)(Pego3)-Hdc (SEQ ID NO: 24) The peptide compositions may also be used to treat COVID-19.

### CROSS-REFERENCES TO RELATED APPLICATIONS

This application is a continuation-in-part and claims benefit of PCT Application No. PCT/US22/75356 filed Aug. 23, 2022, which claims benefit of U.S. application Ser. No. 17/409,642 filed Aug. 23, 2021, the specifications of which are incorporated herein in their entirety by references.

This application is also a continuation-in-part and claims benefit of U.S. application Ser. No. 17/409,642, which is a continuation-in-part and claims benefit of U.S. application Ser. No. 16/093,063, filed Oct. 11, 2018, now U.S. Pat. No. 11,110,152 which is a 371 application of PCT Application No. PCT/US2017/026889, filed Apr. 11, 2017, which claims benefit of U.S. Provisional Application No. 62/320,950, filed Apr. 11, 2016, the specifications of which are incorporated herein in their entirety by references.

U.S. application Ser. No. 17/409,642 is a continuation-in-part and claims benefit of PCT Application No. PCT/US2021/026411, filed Apr. 8, 2021, which claims benefit of U.S. Provisional Patent Application No. 63/006,831, filed Apr. 8, 2020, the specifications of which are incorporated herein in their entirety by references.

### STATEMENT REGARDING FEDERALLY SPONSORED RESEARCH OR DEVELOPMENT

This invention was made with government support under Grant Nos. AI081672, HL125602, HL152942, and AI125357 awarded by National Institutes of Health. The government has certain rights in the invention.

### REFERENCE TO AN ELECTRONIC SEQUENCE LISTING

The contents of the electronic sequence listing (UNIA 2121 CIP2_V2.xml; Size: 41,384 bytes; and Date of Creation: Feb. 22, 2024) is herein incorporated by reference in its entirety.

### FIELD OF THE INVENTION

Provided herein are compositions and methods for treating and preventing lung disease. In particular, provided herein are SP-A peptides and uses thereof in the treatment and prevention of lung disease (e.g., inflammatory lung disease (e.g., asthma)).

### BACKGROUND OF THE INVENTION

Asthma is the most common respiratory disease in both children and adults and affects 10% of the world's population, 25 million people in the US alone. Asthma is a chronic syndrome characterized by airway hyperresponsiveness, inflammation, and intermittent respiratory symptoms. The healthcare burden of asthma is significant, amounting to $81 billion in expenditures yearly in the US when direct care healthcare costs and lost productivity are considered. Despite the considerable costs and increasing prevalence, asthma remains poorly understood and difficult to manage due to the heterogeneity of the disease.

A significant cause of morbidity and mortality in asthma is an acute exacerbation, which can lead to airway injury, remodeling, lung function decline, and death. Most exacerbations are caused by respiratory infections (e.g., rhinovirus or Mycoplasma pneumoniae), and the response to these infections is complex, involving both the innate and adaptive immune systems. In severe asthmatics, exacerbations are associated with accelerated lung function decline. As reduced lung function is a risk factor for severe exacerbations, this vicious cycle can promote an exacerbation-prone phenotype of asthma. Thus, an understanding of the mechanisms driving asthma exacerbations has been a critical barrier to progress in the understanding of asthma pathobiology. The host response in an asthma exacerbation is complex, involving both the innate and adaptive immune systems. Of available treatments, there is no current innate immune modulator for the treatment of asthma, and those directed toward the adaptive immune system have not controlled the disease. Thus, there is a significant need to develop new therapies directed toward the innate response in the treatment of asthma and other inflammatory lung diseases.

### BRIEF SUMMARY OF THE INVENTION

It is an objective of the present invention to provide compositions and methods that allow for the treatment and prevention of lung disease (e.g., inflammatory lung disease (e.g., asthma)), as specified in the independent claims. Embodiments of the invention are given in the dependent claims. Embodiments of the present invention can be freely combined with each other if they are not mutually exclusive.

Surfactant Protein A (SP-A) is a secreted lipoprotein complex. SP-A is an innate immune modulator that is produced and secreted by several types of lung cells (alveolar type II cells, airway club cells, and submucosal gland cells) and serves as the first line of defense against inhaled insults throughout the upper and lower airway (e.g., infectious and/or environmental insults). It acts as a regulator of pathogen phagocytosis and inflammatory processes in the lung. Mature SP-A is a hetero-oligomeric product derived from SP-A1 and SP-A2 genes.

Experiments described herein demonstrated that, in asthma patients, the SP-A2 Gln223Lys (i.e., Q223K) allele, within the SP-A wild type sequence (SEQ ID NO: 1), is associated with decreased lung function, decreased asthma control, and increased BAL and serum eosinophilia. Thus, SP-A is a key regulator of eosinophil degranulation and survival, as well as mucin secretion and type 2 inflammation, and may thereby significantly influence asthma severity. In vitro studies with isolated eosinophils, SP-A deficient mice, and SP-A containing the specific SP-A allele of interest oligomers (e.g., purified peptides described herein comprising SEQ ID NO: 9), found that SP-A directly stimulates eosinophil apoptosis, and this effect can be recapitulated by specific SP-A peptides and that SP-A allelic variants differentially modulate eosinophil responses.

| | |
|---|---|
| SEQ ID NO: 1 | MWLCPLALNLILMAASGAACEVKDVCVGSPGIPG |
| Human Wild | TPGSHGLPGRDGRDGVKGDPGPPGPMGPPGETPC |
| Type amino | PPGNNGLPGAPGVPGERGEKGEAGERGPPGLPAH |
| acid sequence | LDEELQATLHDFRHQILQTRGALSLQGSIMTVGE |
| for SP-A | KVFSSNGQSITFDAIQEACARAGGRIAVPRNPEE |
| | NEAIASFVKKYNTYAYVGLTEGPSPGDFRYSDGT |
| | PVNYTNWYRGEPAGRGKEQCVEMYTDGQWNDRNC |
| | LYSRLTICEF |

Without wishing to limit the present invention to any theories or mechanisms it is believed that SP-A encounters eosinophils in the bronchoalveolar compartment and is a critical regulator of their apoptosis during the resolution phase of inflammatory processes. As described herein, SP-A plays a role in directly inducing apoptosis signaling pathways in eosinophils, which results in the attenuation of allergic phenotypes such as mucin production and eosinophilia. SP-A attenuates mucin and IL-6 induced by IL-13 in airway epithelial cells obtained from asthmatic subjects with allergic or type 2 asthma.

The present invention may feature composition comprising a surfactant protein A (SP-A) peptide analogue comprising a purified peptide comprising an amino acid sequence of KEQCVE (SEQ ID NO: 9). For example, the present invention may feature a composition comprising a purified peptide comprising an amino acid sequence of Ac-WGKEQCVE(Nle)(Pego3)-Hdc (SEQ ID NO: 24). In some embodiments, the present invention features a pharmaceutical composition comprising a purified peptide comprising an amino acid sequence of Ac-WGKEQCVE(Nle)(Pego3)-Hdc (SEQ ID NO: 24). In some embodiments, the pharmaceutical composition may further comprises a pharmaceutically acceptable carrier. The compositions described herein may be in a preparation for aerosolization, subcutaneous injection, or pulmonary delivery.

In some embodiments, the present invention may feature a system comprising the composition (e.g., a pharmaceutical composition) comprising a purified peptide comprising an amino acid sequence of Ac-WGKEQCVE(Nle)(Pego3)-Hdc (SEQ ID NO: 24) and a device for pulmonary delivery of said composition. In other embodiments, the present invention may feature a system comprising the composition (e.g., a pharmaceutical composition) comprising a purified peptide comprising an amino acid sequence of KEQCVE (SEQ ID NO: 9) and a device for pulmonary delivery of said composition. Non-limiting examples of devices for pulmonary delivery include a metered dose inhaler or a nebulizer.

The present invention may also feature compositions and methods for treating and preventing lung diseases (e.g., inflammatory lung diseases). In particular, provided herein are SP-A peptides and uses thereof in the treatment and prevention of lung disease (e.g., asthma). In some embodiments, the present invention features methods of treating inflammatory lung diseases in a subject in need thereof. The method may comprise administering a therapeutically effective amount of any one of the compositions (e.g., purified peptides) as described herein to the subject. The present invention may further feature a purified peptide comprising an amino acid sequence of KEQCVE (SEQ ID NO: 9) for use in a method of treating an inflammatory lung disease in a subject in need thereof.

In some embodiments, the present invention features methods and compositions (e.g., pharmaceutical compositions) for enhancing (e.g., increasing) SP-A activity in a cell. The composition (e.g., a pharmaceutical composition) may comprise any one of the purified peptides as described herein and a pharmaceutical carrier. In some embodiments, the composition is in a preparation for aerosolization. The method may comprise delivering any one of the compositions described herein to a cell (e.g., a lung cell or a nasal cell).

Further embodiments provide a system comprising: a) any one of the compositions described herein; and b) a device for pulmonary delivery of the composition. In some embodiments, the device is a metered dose inhaler.

One of the unique and inventive technical features of the present invention is the use of an amino acid sequence peptide comprising KEQCVE (SEQ ID NO: 9) (i.e., the 6-mer). Without wishing to limit the invention to any theory or mechanism, it is believed that the technical feature of the present invention advantageously provides for inhaled delivery. Additionally, the inclusion of the 6-mer (i.e., KEQCVE (SEQ ID NO: 9)) is necessary for the activity of the larger peptidomimetic (i.e., 10-mers or 20-mers). Additionally, the smaller (i.e., the 6-mer) peptide deposits more deeply in the lungs (i.e., the peptide is able to make it further into the lungs) compared to the larger peptides. Furthermore, the peptide described herein enables the replacement and/or augmentation of SP-A inside the lungs. None of the presently known prior references or work has the unique, inventive technical feature of the present invention.

Of available treatments, there is no current innate immune modulator for the treatment of asthma, and those directed toward the adaptive immune system have not controlled the disease. Thus, there is a significant need to develop new therapies directed toward the innate response in the treatment of asthma and other inflammatory lung diseases. We have discovered small molecules that mimic the effect of Surfactant Protein A (SP-A). SP-A is a natural component of the lung lining fluid and serves as the first line of defense. Some asthma patients either have no SP-A or damaged SP-A. Full-Length SP-A delivered directly to the lungs is not feasible due to its large size and complex structure. We first developed a series of 10-20 amino acid peptides derived from the lectin domain of SP-A2 in order to determine the specific region of activity. Findings described herein demonstrate that 10-20 amino acid SP-A peptides reduce airway constriction, a fundamental characteristic of asthma, in two different preclinical mouse models of asthma

Furthermore, the inventive technical features of the present invention contributed to a surprising result. For example, the peptidomimetics that include the 6-mer (i.e., KEQCVE (SEQ ID NO: 9)) impact (i.e., decreases) both eosinophil viability and STAT-signaling, which would ultimately be a benefit to a patient with asthma. Additionally, the peptidomimetics that include the 6-mer (i.e., KEQCVE (SEQ ID NO: 9)) have activity in both males and females in preclinical animal testing. Furthermore, the peptidomimetics that include the 6-mer (i.e., KEQCVE (SEQ ID NO: 9)) work in two phases: (1) an acute phase to reduce airway mucus production and hyperreactivity to methacholine and (2) a late phase to clear the lung of inflammatory eosinophils and neutrophils. Finally, the peptidomimetics, including the 6-mer (i.e., KEQCVE (SEQ ID NO: 9)), work over a longer duration of time; for example, one dose can last in effectiveness for 7-10 days. In human airway cells, the peptidomimetics also reduce mucous and pro-inflammatory mediators

Any feature or combination of features described herein are included within the scope of the present invention, provided that the features included in any such combination are not mutually inconsistent as will be apparent from the context, this specification, and the knowledge of one of ordinary skills in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a non-limiting overview of the how the 10-mer (i.e., SEQ ID NO: 4) and/or the 20-mer (i.e., SEQ ID NO: 8) SP-A peptides, as well as, the other SP-A peptides described herein reduce airway constriction, a fundamental characteristic of asthma, in two different preclinical mouse models of asthma. As shown in FIG. 1, the mechanisms of protective action discovered herein are due to 1) direct interaction with eosinophils, a critical inflammatory cell in asthma, to induce apoptosis and promote their resolution from the airway and 2) direct interaction with epithelial cells, which line the lung and participate in the inflammatory process, to inhibit mucin production.

FIGS. 2A and 2B show the genetic variation in SP-A2 is associated with changes in lung function and asthma control. The percent predicted FEV1 and asthma control questionnaire score in a cohort of 53 asthmatic subjects stratified by alleles of rs1965708 (Gln223Lys) of the SP-A2 gene. The asthmatic subjects with the 223K/K genotype demonstrate significantly worse asthma control (right panel) and lower lung function (left panel) as compared to the heterozygotes for 223Q/K and major allele (homozygosity for 223Q/Q) genotype. *p<0.05 compared to Q/Q.

FIGS. 3A, 3B, 3C, 3D, 3E, 3F, 3G, and 3H show genetic variation in SP-A2 determines the extent of protection against IL-13 induced inflammation in humanized SP-A transgenic mice. BAL cells from IL-13 challenged mice (FIG. 3A), which consisted of macrophages (FIG. 3B), neutrophils (FIG. 3C), and eosinophils (FIG. 3D). FIG. 3E shows PAS-scored lung histology from IL-13 challenged mice. N=12 WT; 15 SP-A−/−; 8 SP-A223Q/Q; 12 SP-A223K/K per group from 3 separate experimental repeats. *p<0.05, **p<0.01, ***p<0.001. ****p<0.0001 by One-way ANOVA with Dunnett's test for multiple comparisons. FIG. 3F shows representative PAS images of each genotype treated with IL-13. FIGS. 3G and 3H show Stat3 phosphorylation by Western blot and densitometry from representative lung samples; *p<0.05 by One-Way ANOVA with Tukey's multiple comparisons.

FIGS. 4A and 4B show genetic variation in SP-A2 determines the extent of protection against IL-13-induced inflammation from bronchial epithelial cells from asthmatic participants. FIG. 4A shows MUC5AC RNA expression from bronchial epithelial cells (n=3 normal, n=3 asthma) grown at air-liquid interface (ALI) and treated with IL-13 for 5 days in the presence or absence of full-length recombinant SP-A2(223K) (20 μg/ml) or SP-A2(223Q) (20 μg/ml) that were added 30 min prior to the challenge. After standardization to the housekeeper gene, data are displayed as fold relative to the non-IL-13 challenged control for each respective patient set, with the standard deviation shown. Average fold change and standard deviation are displayed. *p<0.05. FIG. 4B shows genetic variants of SP-A2 that differ only at position 223 (Q and K) were examined for relative binding to IL-13 relative to extracted human oligomeric control SP-A.

FIGS. 5A, 5B, 5C, and 5D show that HDM-challenged SP-A deficient mice treated with SP-A peptides have reduced hallmarks of inflammation. FIG. 5A shows SP-A deficient mice challenged intra-nasally with HDM on days 0, 7, and 14. On day 15, mice were divided into groups and given either vehicle or SP-A peptides (10-mer (i.e., SEQ ID NO: 4) or 20-mer (i.e., SEQ ID NO: 8) via oropharyngeal instillation at 25 μg/ml concentration (˜1 mg/kg body weight). On day 19, mice were sacrificed, and eosinophils in the BAL (FIG. 5B) and mucin production (FIG. 5C) in lung histological sections (FIG. 5D) were assessed. n=10,10, **p<0.01, ***p<0.001 by One-way Anova for multiple comparisons.

FIGS. 6A, 6B, and 6C show the shortened 10AA peptides reduce mucin production (FIG. 6A), and eosinophilia (FIG. 6B) in mouse HDM model, and reduce Mucin (Muc5AC RNA) in human primary cells (6C).

FIGS. 7A, 7B, 7C, 7D, and 7E show that HDM-challenged WT mice treated with SP-A peptides have reduced sensitivity to methacholine challenge. FIG. 7A shows WT male mice challenged intranasally with HDM on days 0, 7, and 14. On days 1, 8, and 15, mice were divided into groups and given either vehicle or SP-A peptides (10-mer (i.e., SEQ ID NO: 4), 25 μg/ml, ˜1 mg/kg body weight) via oropharyngeal instillation. On day 19, pulmonary function tests during a methacholine challenge were performed while mice were under anesthesia. FIG. 7B shows total airway resistance (Rrs), FIG. 7C shows Newtonian resistance (Rn), FIG. 7D shows total airways Elastance (Ers) and FIG. 7E shows tissue damping that was assessed by Flexivent. Data graphed are mean+/−SEM. n=12,12, *p<0.05, **p<0.01 by t-test at each indicated dose

FIGS. 8A and 8B show SP-A peptide protects against airway hyperresponsiveness (AHR) in an IL-13 model.

FIG. 9 shows IL-13-challenged mice treated with SP-A 10-mer peptide (i.e., SEQ ID NO: 4) have improved lung function. WT male mice were challenged with vehicle (saline) or IL-13 (3.9 μg) by oropharyngeal delivery for 3 consecutive days. Two hours after each IL-13 challenge, mice received either vehicle (saline) or SP-A 10-mer peptide (25 μg/ml; ˜1 mg/kg body weight) via oropharyngeal delivery. Pulmonary function tests were conducted on day 4 on a flexiVent machine (SCIREQ) with the negative pressure-driven forced expiration (NPFE) extension. IL-13 challenge resulted in significantly increased Newtonian resistance (Rn) and decreased forced expiratory volumes (FEV) at 0.05 seconds. Treatment with SP-A 10-mer peptide protected against IL-13 induced increase in Rn and decreases in FEV. Average mean+/−SEM is graphed, n=as shown from 2 independent experiments. *p<0.05, ***p<0.001 by ANOVA for multiple comparisons.

FIG. 10 shows primary human lung epithelial cells treated with SP-A peptides have reduced IL-13 induced MUC5AC gene expression. Primary human bronchial epithelial cells derived from normal and asthmatic participants were incubated for 30 minutes with either 20-mer (i.e., SEQ ID NO: 8) or 10-mer (i.e., SEQ ID NO: 8) SP-A peptide (20 μg/ml) before stimulation with IL-13 (10 ng/ml) for 5 days. When taken together (asthma and normal cells), MUC5AC gene expression was significantly reduced in the SP-A 20-mer treatment group compared with IL-13 alone (p=0.004).

FIGS. 11A, 11B, 11C, and 11D show an assessment of BALF eosinophilia over time. FIG. 11A shows an OVA model of allergic airways. FIG. 11B shows cell distribution in BALF at 24 hours, 3 days, and 5 days post-terminal challenge. FIG. 11C shows a set change in eosinophil frequencies over time. FIG. 11D shows the difference in means at 24 hours and 5 days, unpaired Student's t-test. One-way ANOVA with Bonferroni's correction for multiple comparisons, *p<0.05, **p<0.01 Data (mean+SEM) are from at least two independent experiments with n=3-5 mice/group.

FIGS. 12A, 12B, 12C, 12D, and 12E show assessment of tissue eosinophilia over time. FIG. 12A shows representative bright field images of eosinophils (arrows indicate representative eosinophils) in lung tissue by Sirius red staining (top panel: 40× magnification, bottom panel: 100× magnification) and FIG. 12B shows quantification of eosinophil counts at day 5. FIG. 12C shows a net change in eosinophil frequencies over time. FIG. 12D shows the difference in means at 24 hours and 5 days, unpaired Student's t-test, #p<0.05, **p<0.01 FIG. 12E shows eosinophil-associated ribonuclease (EAR) mRNA in lung tissue at 5 days post-terminal challenge. One-way ANOVA with Bonferroni's correction for multiple comparisons, *p<0.05. Data (mean+SEM) are from at least two independent experiments with n=3-5 mice/group.

FIGS. 13A, 13B, 13C, and 13D show the evaluation of the ability of SP-A to induce eosinophil apoptosis in mouse and human eosinophils in vitro. FIG. 13A shows a time course of viability assessed by Trypan Blue and FIG. 13B shows real-time cell analyzer (RTCA) tracing and dose-response of in vitro stimulation of mouse eosinophils by SP-A, AUC=area under the curve. FIG. 13C representative flow diagrams of human eosinophil apoptosis and cell death by Annexin V and PI and quantification after 16 hours incubation with SP-A; live=Annexin V−, PI−, early apoptosis=Annexin V+, PI−, late apoptosis/dead=Annexin V+, PI+FIG. 13D shows densitometry of caspase-3 by Western blot of mouse eosinophils standardized to non-treated control. ANOVA with correction for multiple comparisons, *p<0.05, **p<0.01, ***p<0.001, ****p<0.0001. Data (mean+SEM) are from at least two independent experiments with n=2-3 replicates/treatments.

FIGS. 14A, 14B, and 14C show the evaluation of the effect of exogenous SP-A administration on eosinophils in SP-A deficient mice after the OVA challenge. FIG. 14A shows a schematic of the OVA challenge and SP-A rescue. FIG. 14B shows representative flow diagrams of eosinophil apoptosis and cell death by Annexin V and PI. FIG. 14C shows total live eosinophil counts in BALF 5 days post-terminal challenge. *p<0.05. Data (mean+SEM) are representative of two independent experiments with n=5 mice/group.

FIGS. 15A and 15B show the analysis of inflammation in SP-A2 humanized mice in the Ova model. Mice were sensitized and challenged in the Ova model and BAL cellularity (left panel) was assessed 24 hrs post challenge, and mucin production (right panel) was assessed 7 days post-challenge. The presence of human SP-A 223Q in mice resulted in more protection as determined by less eosinophilia and mucin production as compared to SP-A−/− mice. SP-A 223Q expressing mice had similar BAL eosinophilia and mucin production as compared to WT control mice (that have normal mouse SP-A) after the Ova challenge.

FIGS. 16A and 16B show the analysis of inflammation in mice that receive “therapeutic” SP-A peptides in the HDM model. Mice were sensitized and challenged in the HDM model according to common methods. Twenty-four hours after the last challenge, mice received either a vehicle, a 20-mer, or a 10-mer, that encompassed the active site containing 223Q. BAL cellularity (left panel) and mucin production (right panel) were assessed 7 days post-challenge to assess the role of SP-A on allergic airway resolution. SP-A KO mice have significantly enhanced BAL eosinophilia as compared to WT mice. Both 223Q and 223K mice are somewhat protected in the acute phase of this model.

FIG. 17 shows SP-A 223Q 10-mer peptides significantly suppress MUCSAC expression following IL-13 exposure in human airway epithelial cells. Airway epithelial cells cultured at an air-liquid interface from two asthmatic participants were exposed to IL-13 alone or IL-13 plus SP-A2 peptide that includes the Gln at position 223 in the lectin domain (223Q). Full-length oligomeric SP-A that is homozygous at position 223Q/Q was used as a positive control. After 48 hours of incubation, MUCSAC expression was determined by RT-PCR.

FIG. 18 shows SP-A is significantly decreased in asthmatic patients who are obese. This is potentially a target group for SP-A peptide therapy.

FIG. 19 shows that in human airway epithelial cells taken from asthma patients, the peptides and lead peptidomimetic described herein (i.e., SEQ ID NO: 4 and SEQ ID NO: 8 (peptides); and SEQ ID NO: 12 (peptidomimetic)) reduce a key signaling pathway that is activated in asthma-STAT3. Activation of Stat3 signaling leads to airway inflammation and mucus production, which makes asthma worse. Reducing this key signaling pathway by 40-50% and/or now 80% with top candidates (e.g., SEQ ID NO: 12), would translate to significant protection in an asthma patient's lungs by reducing symptoms of asthma. Specifically, the bottom graph shows peptidomimetic lead (i.e., SEQ ID NO: 12) reduces IL-13 stimulated Stat-3 signaling in airway epithelial cells from asthma patients. Bronchial epithelial cells were cultured at an air-liquid interface and differentiated for 14 days. Cells were treated basolateral with peptidomimetic lead 867 (i.e., SEQ ID NO: 12) at increasing doses for 1 hr, prior to basolateral stimulation with IL-13 (50 ng/ml) for 30 min. Total cell lysates were analyzed for STAT3 phosphorylation by Western blot compared with total STAT3 and ß-actin.

FIG. 20 shows a peptidomimetic lead (e.g., SEQ ID NO: 25 or C892) reduces IL-6 stimulated STAT-3 signaling in HEK reporter cells. Cells were pretreated with C892 for 30 min at increasing concentrations prior to stimulation with IL-6 (16 ng/ml) for 18 hrs. STAT-3 activation measured by cell fluorescence was read after one minute of exposure to the substrate on the Clariostar machine. n=3 replicates per condition.

FIG. 21 shows a peptidomimetic lead (e.g., SEQ ID NO: 12 or C867) reduces MUC5AC expression from airway epithelial cells from 4 participants with type 2 asthma. Cells obtained by bronchoscopy were cultured at an air-liquid interface for 14 days. At 14 days, the cells differentiate, express cilia, and produce mucus. Some cells were pretreated with C867 (13.08 UM) for one hour prior to stimulation with IL-13 (10 ng/ml), a significant stimulus for mucus and mucin gene expression, for five days. MUC5AC gene expression was significantly reduced by 42% (p=0.01).

FIG. 22 shows a peptidomimetic lead (e.g., SEQ ID NO: 25 or C892) reduces bronchoconstriction to methacholine challenge in an asthma model. WT C57BL/6 female mice were treated with HDM on days 0, 7, 14, and 24 hrs after each challenge; some mice received lead compounds (from Table 1). AHR to methacholine was performed on day 16. *p<0.05 by ANOVA, HDM vs Saline; HDM+Lead compound was not different from saline controls.

FIG. 23 shows peptidomimetic lead reduces IL-6 stimulated STAT-3 signaling in HEK reporter cells compared to full length SP-A. Cells were pre-treated with either full-length SP-A or the lead peptide for 30 min at increasing concentrations prior to stimulation with IL-6 (16 ng/ml) for 18 hrs. STAT-3 activation measured by cell fluorescence was read after one minute of exposure to the substrate on the Clariostar machine. n=3 replicates per condition.

FIG. 24 shows peptidomimetic lead (i.e., compound 939, i.e., SEQ ID NO: 23) reduces IL-13 stimulated Stat-3 signaling in airway epithelial cells from asthma patients. Bronchial epithelial cells were cultured at an air-liquid interface and differentiated for 14 days. Cells were treated basolateral with lead mimetic at increasing doses for 1 hr, prior to basolateral stimulation with IL-13 (50 ng/ml) for 30 min. Total cell lysates were analyzed for STAT3 phosphorylation by Western blot.

FIG. 25 shows the structures of SEQ ID NO: 23 comprising a small oligoethyleneglycol linker, Pego. Specifically, SEQ ID NO: 23 comprises 3 Pego units (i.e., Pego3), each with 3 ethyleneglycols connected via diglyme diacid, MW 230 per unit. At the C-terminal, there is now a lipid attachment Hdc.

FIG. 26 shows SP-A binding to HEK293T lysates over-expressing ACE2. SP-A coated plates were incubated with lysates from 293T cells overexpressing ACE2 in varying concentrations in the presence (solid line) or absence of Ca2+(dotted line). Binding was detected with human anti-ACE2 by absorbance at 450 nm wavelength and was read by a plate reader. Non-transfected 293T cell lysates (dashed line) were tested as a negative control. n=3 experiments.

FIG. 27 shows SP-A 20-mer peptides compete for binding to ACE overexpressing cells. Full-length SP-A or SP-A 20-mers competed for (inhibited) ACE2 binding to plate-bound SP-A. 20-mer scrambled (SCR) peptide had little to no effect. n=3 experiments.

FIGS. 28A and 28B show full-length SP-A reduces the binding of Spike protein to cells overexpressing ACE2.

FIG. 29 shows SP-A reduced transduction of S1 protein-pseudotyped lentiviral particles in vitro to cells overexpressing ACE2.

FIG. 30 shows SP-A attenuates SARS-COV-2 infection in alveolar organoids. Pre-treatment of alveolar organoids with SP-A significantly reduced SARS-COV-2 N gene expression. Data are represented as fold change±SEM relative to the CoV2 sample. No N gene expression was detected in MOCK samples. Data were analyzed using One-Way ANOVA with Tukey's post-hoc test ****p<0.0001. n=3 technical replicates.

FIG. 31 shows SP-A 20-mer peptides inhibit ACE-2/SPIKE mediated pseudoviral entry.

### DEFINITIONS

The terms “polypeptide” and “protein” are used interchangeably to refer to a polymer of amino acid residues, comprising natural or non-natural amino acid residues, and are not limited to a minimum length. Thus, peptides, oligopeptides, dimers, multimers, and the like are included within the definition. Both full-length proteins and fragments thereof are encompassed by the definition. The terms also include post-translational modifications of the polypeptide, including, for example, glycosylation, sialylation, acetylation, and phosphorylation. Furthermore, a “polypeptide” herein also refers to a modified protein, such as single or multiple amino acid residue deletions, additions, and substitutions to the native sequence, as long as the protein maintains a desired activity. For example, a serine residue may be substituted to eliminate a single reactive cysteine or to remove disulfide bonding or a conservative amino acid substitution may be made to eliminate a cleavage site. These modifications may be deliberate, as through site-directed mutagenesis, or may be accidental, such as through mutations of hosts, which produce the proteins or errors due to polymerase chain reaction (PCR) amplification.

As used herein, the term “peptide” refers to a short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (synthetic) sequence.

As used herein, the term “wildtype” refers to a non-mutated version of a gene, allele, genotype, polypeptide, phenotype, or a fragment of any of these. It may occur in nature or be produced recombinantly. As used herein, the term “variant” refers to a nucleic acid molecule or polypeptide that differs from a referent nucleic acid molecule or polypeptide by single or multiple amino acid substitutions, deletions, and/or additions and substantially retains at least one biological activity of the referent nucleic acid molecule or polypeptide.

The terms “peptide mimetic” or “peptidomimetic” refer to a peptide-like molecule that emulates a sequence derived from a protein or peptide. A peptide mimetic or peptidomimetic may contain amino acids and/or non-amino acid components. Examples of peptidomimetics include chemically modified peptides, peptoids (side chains are appended to the nitrogen atom of the peptide backbone, rather than to the α-carbons), P-peptides (amino group bonded to the β carbon rather than the α carbon), etc.

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another: (1) Alanine (A) and Glycine (G); (2) Aspartic acid (D) and Glutamic acid (E); (3) Asparagine (N) and Glutamine (Q); (4) Arginine (R) and Lysine (K); (5) Isoleucine (I), Leucine (L), Methionine (M), and Valine (V); (6) Phenylalanine (F), Tyrosine (Y), and Tryptophan (W); (7) Serine (S) and Threonine (T); and (8) Cysteine (C) and Methionine (M).

Naturally occurring residues may be divided into classes based on common side chain properties, for example: polar positive (histidine (H), lysine (K), and arginine (R)); polar negative (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include but are not limited to, peptidomimetics and other reversed or inverted forms of amino acid moieties. Embodiments herein may, in some embodiments, be limited to natural amino acids, non-natural amino acids, and/or amino acid analogs. Non-conservative substitutions may involve the exchange of a member of one class for a member from another class.

As used herein, the term “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Subject,” “individual,” “host,” “animal,” and “patient” are used interchangeably herein to refer to mammals, including, but not limited to, rodents, simians, humans, felines, canines, equines, bovines, porcines, ovines, caprines, mammalian laboratory animals, mammalian farm animals, mammalian sport animals, and mammalian pets.

As used herein, the terms “administration” and “administering” refer to the act of giving a drug, prodrug, or other agent, or therapeutic treatment (e.g., SP-A peptide) to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “co-administration” and “co-administering” refer to the administration of at least two agent(s) (e.g., multiple SP-A peptides or an SP-A peptide and another therapeutic agent) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

“Treatment,” as used herein, covers any administration or application of a therapeutic for disease in a mammal, including a human, and includes inhibiting the disease, arresting its development, or relieving the disease, for example, by causing regression, or restoring or repairing a lost, missing, or defective function; or stimulating an inefficient process.

A “pharmaceutically acceptable carrier” refers to a non-toxic solid, semisolid, or liquid filler, diluent, encapsulating material, formulation auxiliary, or carrier conventional in the art for use with a therapeutic agent for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause an injection site reaction.

As used herein, an “inflammatory lung disease” refers to any lung disease with high levels of inflammation where leukocytes, including neutrophils and eosinophils, are the predominant immune cell infiltrating the lung. Inflammatory diseases may include but are not limited to asthma, chronic obstructive pulmonary disease (COPD), acute respiratory distress syndrome (ARDS), Coronavirus disease (COVID, e.g., COVID-19), and other respiratory infections.

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting.

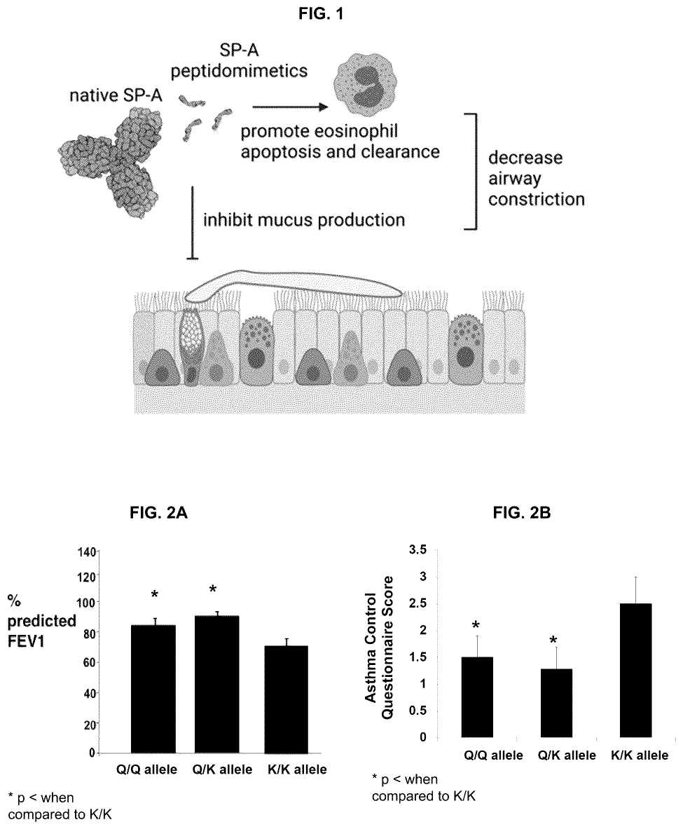

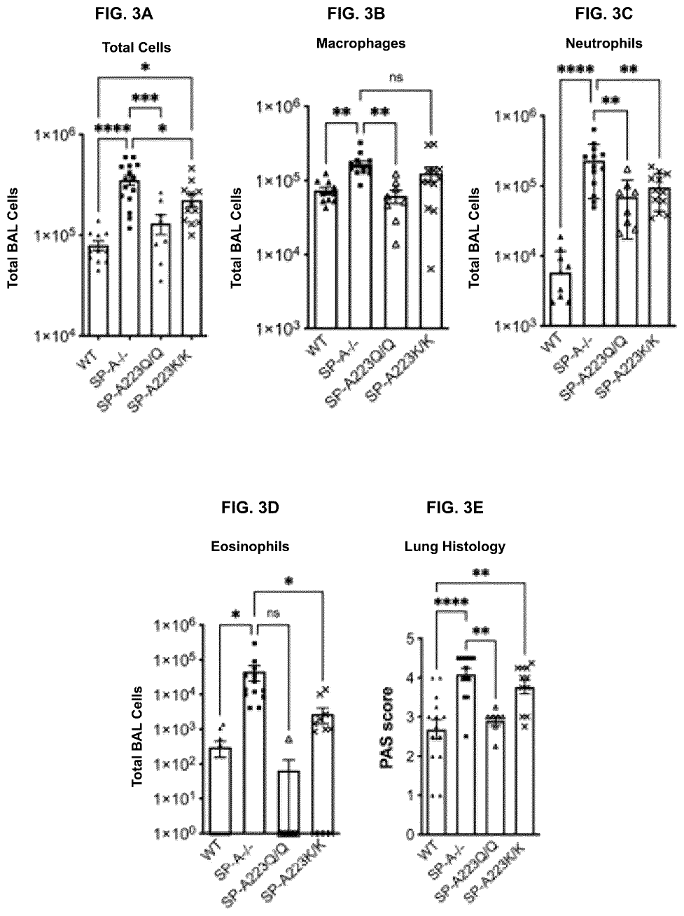

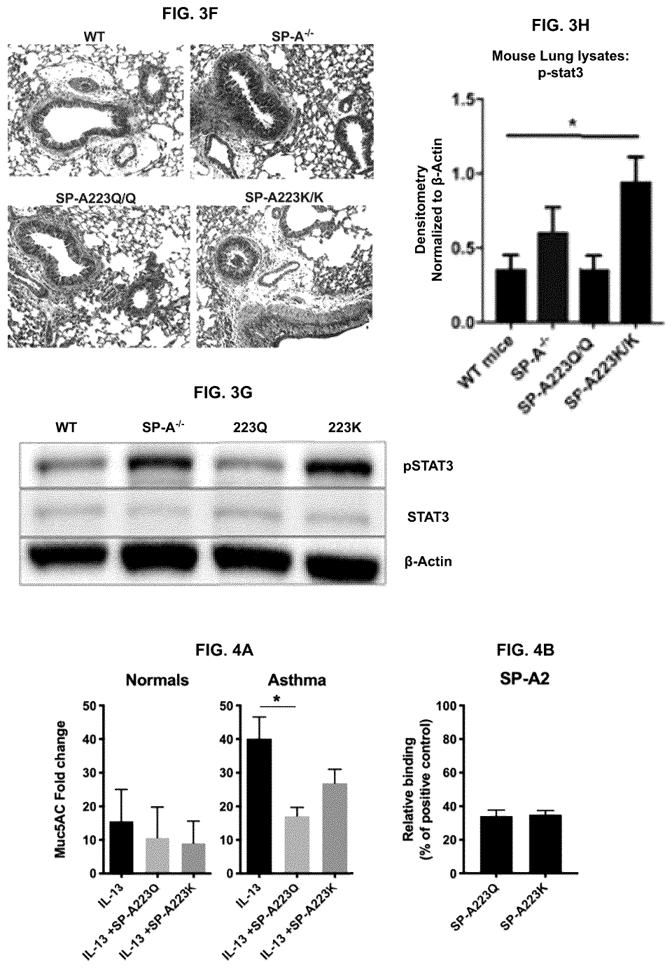

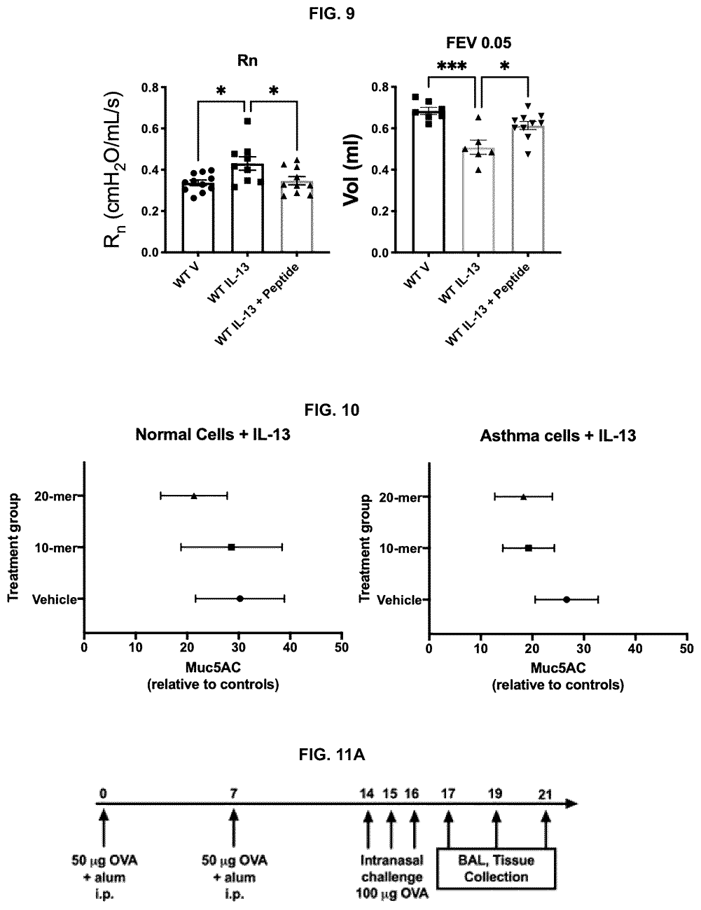

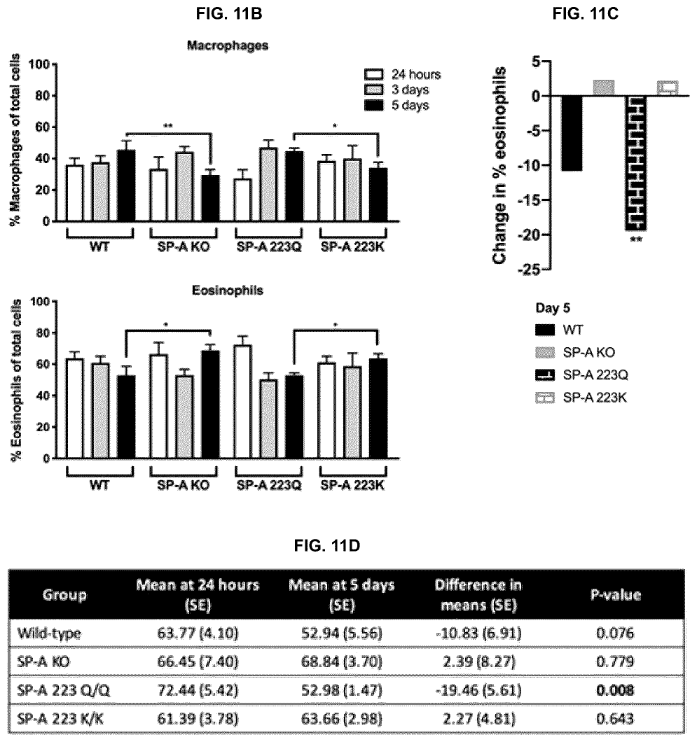

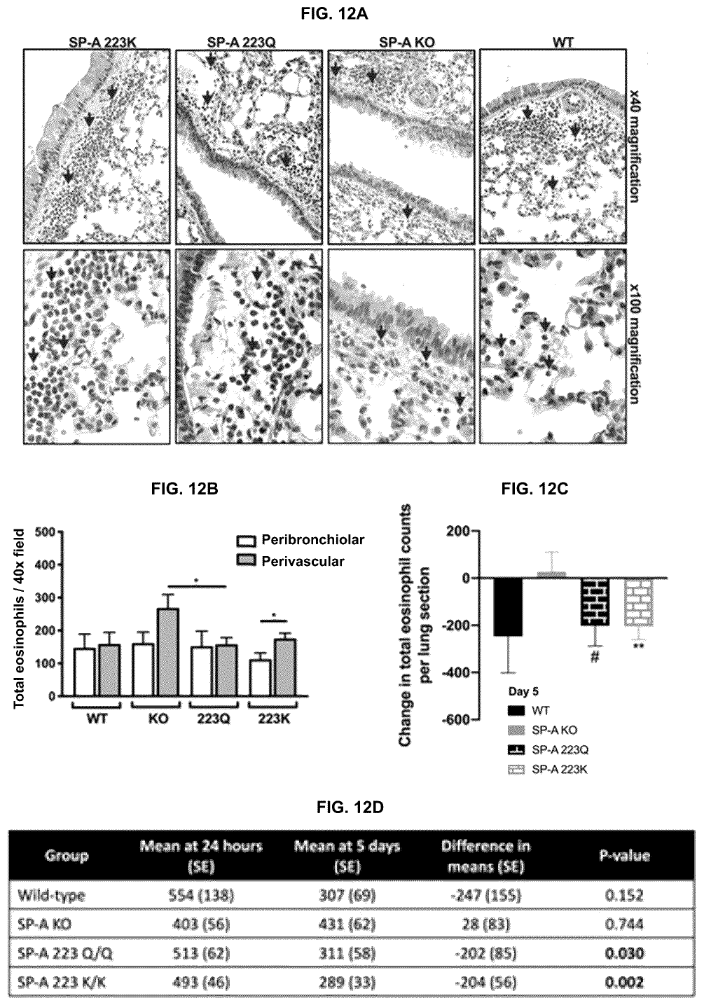

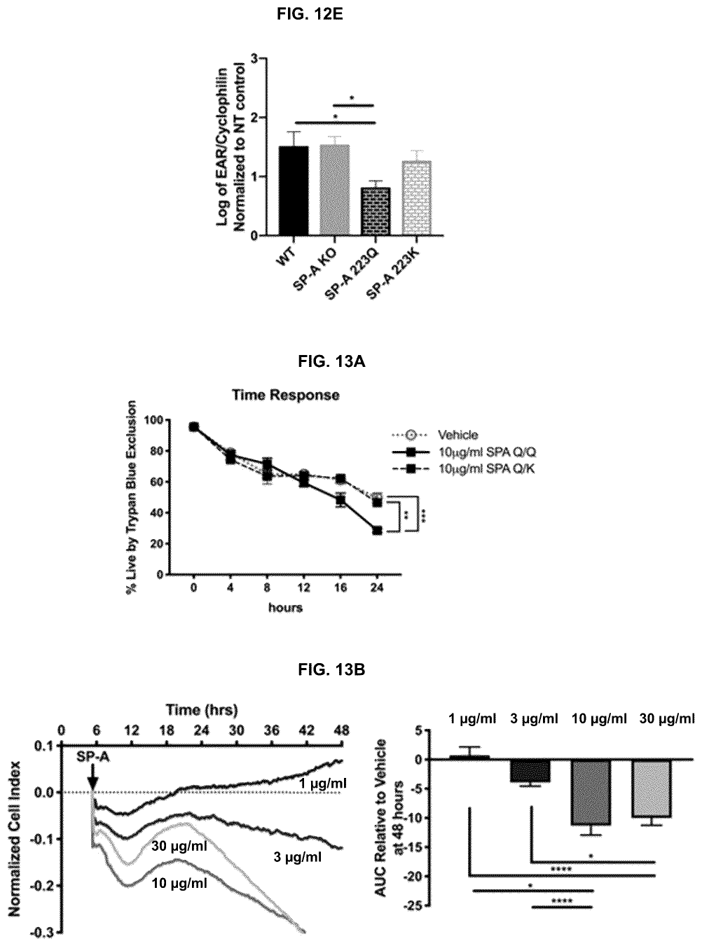

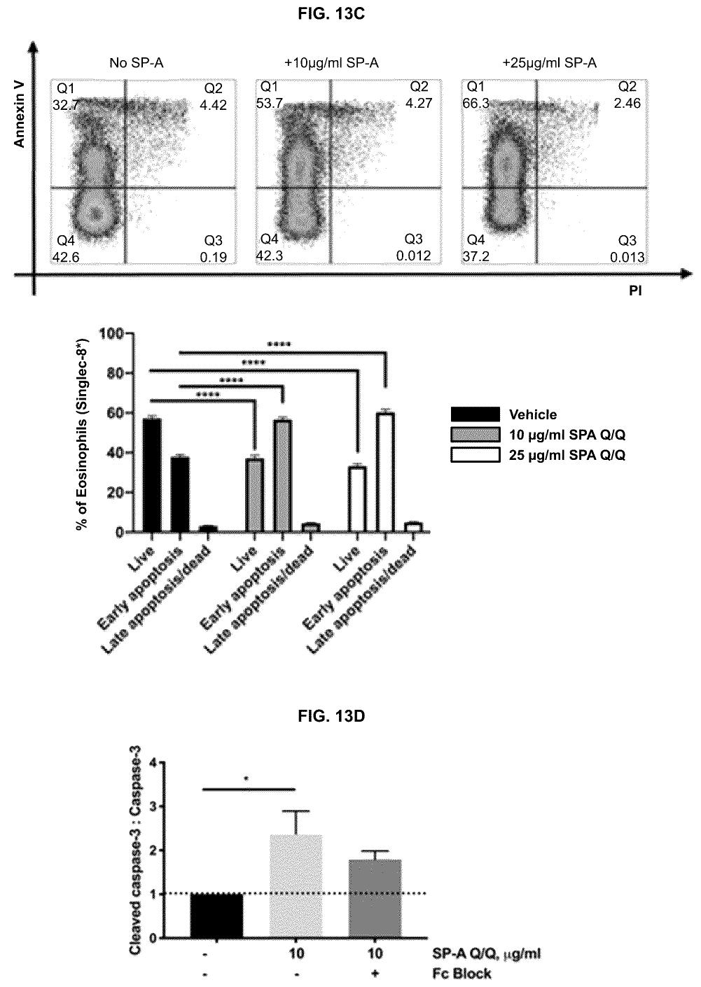

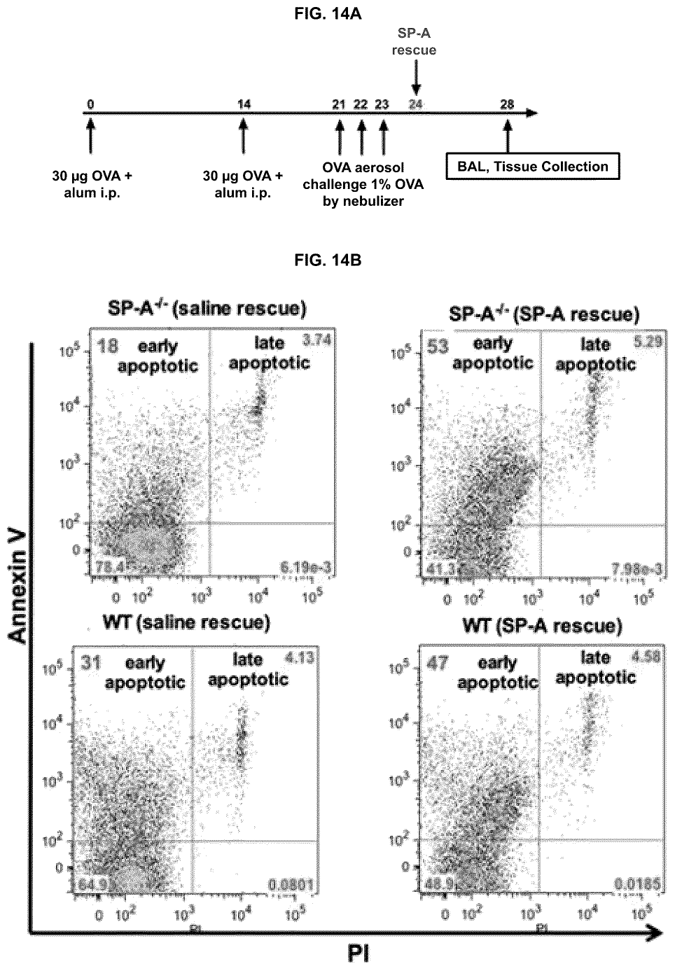

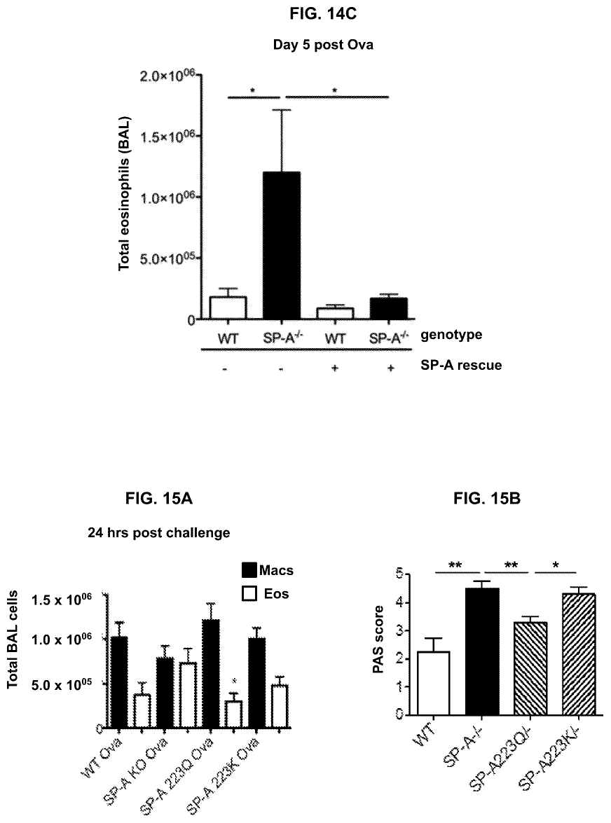

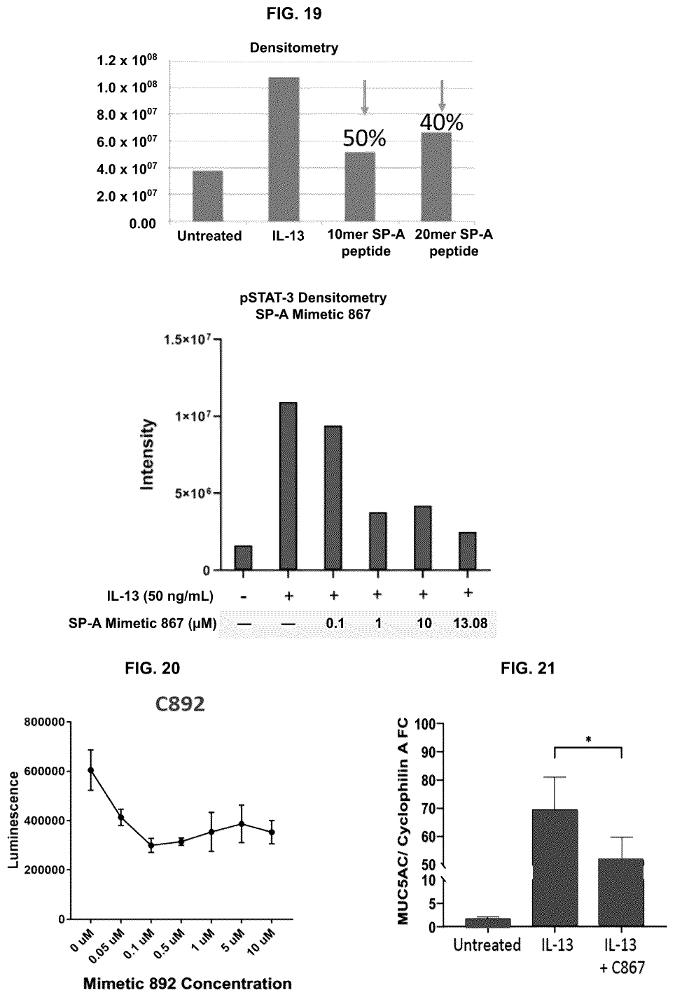

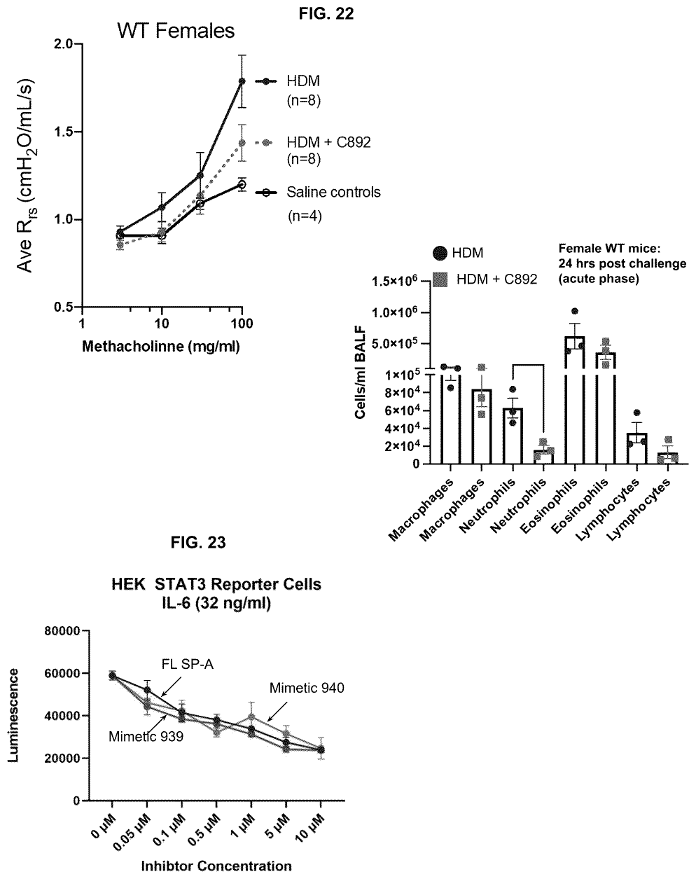

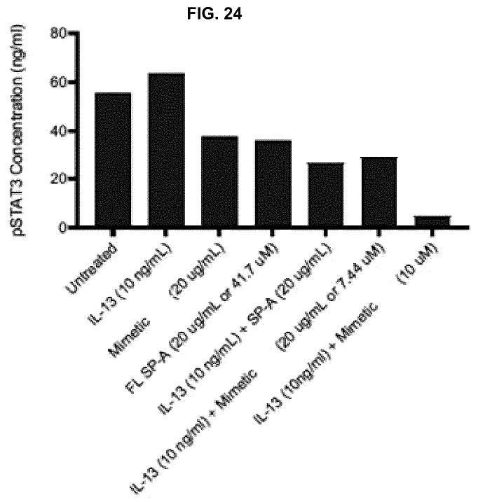

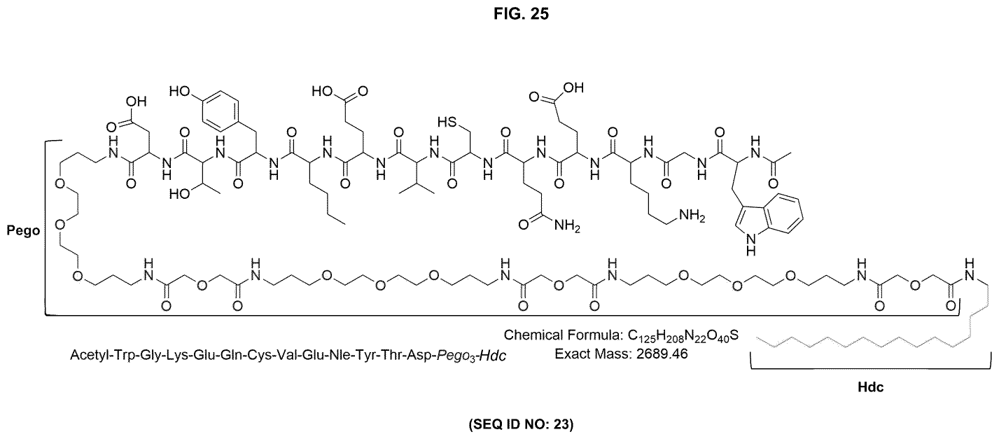

Referring now to FIGS. 1-31, the present invention features compositions and methods for treating and preventing lung disease (e.g., inflammatory lung disease). In particular, provided herein are SP-A peptides and uses thereof in the treatment and prevention of lung disease (e.g., inflammatory lung disease (e.g., asthma)).

The present invention features compositions and methods for treating and preventing lung disease (e.g., inflammatory lung disease) using peptides whose sequence is derived from an active region of endogenous human SP-A and comprises the major Q allele at position 223 of the SP-A2 peptide. For example, in some embodiments, a composition comprising a peptide comprising, consisting essentially of, or consisting of an amino acid sequence selected from, for example, SEQ ID NO: 2, SEQ ID NO: 3, SEQ ID NO: 4, SEQ ID NO: 5, SEQ ID NO: 6, SEQ ID NO: 7, SEQ ID NO: 8, or peptides with at least 90% (e.g., 91%, 92%, 93%, 94%, 95%, 96%, 97%, 98%, 99%, or 100%) identity to the peptides is provided. In certain embodiments, a composition comprising a peptide comprising, consisting essentially of, or consisting of an amino acid sequence selected from, for example, SEQ ID NO: 21, SEQ ID NO: 22, SEQ ID NO: 23, SEQ ID NO: 24, or peptides with at least 90% (e.g., 91%, 92%, 93%, 94%, 95%, 96%, 97%, 98%, 99%, or 100%) identity to the peptides is provided. In some embodiments, the peptide binds to a receptor selected from, for example, FC (CD16/32), Sirp-alpha, TLR-2, EGFR, or MYADM (myeloid-associated differentiation marker).

In some embodiments, the present invention may feature a method of treating an inflammatory lung disease (e.g., asthma) in a subject in need thereof. The method may comprise administering a therapeutically effective amount of a purified peptide as described herein (e.g., a purified peptide comprising an amino acid sequence of Ac-WGKEQCVE(Nle)(Pego3)-Hdc (SEQ ID NO: 24)) to the subject. In other embodiments, the present invention features a purified peptide comprising an amino acid sequence of Ac-WGKEQCVE(Nle)(Pego3)-Hdc (SEQ ID NO: 24) for use in a method of treating an inflammatory lung disease in a subject in need thereof.

In some embodiments, the present invention may feature a method of treating an inflammatory lung disease (e.g., asthma) in a subject in need thereof. The method may comprise administering a therapeutically effective amount of a purified peptide as described herein (e.g., a purified peptide comprising an amino acid sequence of KEQCVE (SEQ ID NO: 9)) to the subject. In other embodiments, the present invention features a purified peptide comprising an amino acid sequence of KEQCVE (SEQ ID NO: 9) for use in a method of treating an inflammatory lung disease in a subject in need thereof.

The present invention may also feature methods and compositions (e.g., pharmaceutical compositions) for enhancing (e.g., increasing) SP-A activity in a cell. The composition may comprise any of the purified peptides as described herein and a pharmaceutical carrier. In some embodiments, the composition (e.g., the pharmaceutical composition) comprises a purified peptide comprising an amino acid sequence of KEQCVE (SEQ ID NO: 9; e.g., Ac-WGKEQCVE(Nle)(Pego3)-Hdc (SEQ ID NO: 24) and a pharmaceutical carrier. In other embodiments, the composition (e.g., the pharmaceutical composition) comprises a purified peptide comprising an amino acid sequence of KEQCVE(Xaa), (SEQ ID NO: 10) and a pharmaceutical carrier; where n ranges from 4-16 amino acids, and Xaa is any natural or non-natural amino acid. In further embodiments, the composition (e.g., the pharmaceutical composition) comprises a purified peptide comprising an amino acid sequence of (Xaa), KEQCVE(Xaa), (SEQ ID NO: 20) and a pharmaceutical carrier, where n ranges from 1-16 amino acids, and Xaa is any natural or non-natural amino acid. In some embodiments, the composition is in a preparation for aerosolization. The method for enhancing (e.g., increasing) SP-A activity in a cell may comprise delivering a composition comprising a purified peptide as described herein (e.g., a peptide comprising an amino acid sequence of KEQCVE (SEQ ID NO: 9; e.g., Ac-WGKEQCVE(Nle)(Pego3)-Hdc (SEQ ID NO: 24)) to a cell (e.g., a lung cell or a nasal cell).

In some embodiments, n ranges from 0-20 amino acids. In some embodiments, n ranges from 0-15 amino acids. In some embodiments, n ranges from 0-10 amino acids. In some embodiments, n ranges from 0-5 amino acids. In some embodiments, n ranges from 0-1 amino acids. In some embodiments, n ranges from 1-20 amino acids. In some embodiments, n ranges from 1-15 amino acids. In some embodiments, n ranges from 1-10 amino acids. In some embodiments, n ranges from 1-5 amino acids. In some embodiments, n ranges from 4-20 amino acids. In some embodiments, n ranges from 4-15 amino acids. In some embodiments, n ranges from 4-10 amino acids. In some embodiments, n ranges from 4-5 amino acids. In some embodiments, n ranges from 5-20 amino acids. In some embodiments, n ranges from 5-15 amino acids. In some embodiments, n ranges from 5-10 amino acids. In some embodiments, n ranges from 10-20 amino acids. In some embodiments, n ranges from 10-15 amino acids. In some embodiments, n ranges from 15-20 amino acids.

Table 1 shows non-limiting examples of purified peptides that may be used in accordance with the compositions and methods described herein.

| | SEQ |
|---|---|
| | ID |
| Purified Peptide Sequence | NO: |
| PAGRGKEQCV | 2 |
| EMYTDGQWND | 3 |
| KEQCVEMYTD | 4 |
| PAGRGKEKCV | 5 |
| KEKCVEMYTD | 6 |
| PAGRGKEKCVEMYTDGQWND | 7 |
| PAGRGKEQCVEMYTDGQWND | 8 |
| PAGRGKEQCV | 2 |
| EMYTDGQWND | 3 |
| KEQCVE | 9 |
| KEQCVEXn | 10 |
| Ac-KEQCVEMYTD-NH2 | 11 |
| Ac-WGKEQCVEMYTD-NH2 | 12 |
| (Ac-KEQCVEMYTD-NH2)2 | 13 |
| Ac-KEQCVEMYTD-acid | 14 |
| H-KEQCVEMYTD-acid | 15 |
| Ac-KEQCVE-Nle-YTD-NH2 | 16 |
| Ac-WGKEQCVE-Nle-YTD-NH2 | 17 |
| Ac-RGKEQCVE-Nle-YTD-NH2 | 18 |
| C-WGKEQCVE-Nle-YTD-NH2 | 19 |
| (Xaa)nKEQCVE(Xaa)n | 20 |
| Ac-KEQCVEMYTD-acid | 14 |
| H-KEQCVEMYTD-acid | 15 |
| Ac-WGKEQCVE(Nle)YTD(Pego3)-NH2 | 21 |
| Ac-WGKEQCVE(Nle)(Pego3)-NH2 | 22 |
| Ac-WGKEQCVE(Nle)YTD(Pego3)-Hdc | 23 |
| Ac-WGKEQCVE(Nle)(Pego3)-Hdc | 24 |

In some embodiments, the aforementioned peptides (i.e., the peptides listed in Table 1) comprise, consist essentially of, or consists of an amino acid sequence of at least 80% (e.g., 81%, 82%, 83%, 84%, 85%, 86%, 87%, 88%, 89%, or 90%) identity to the peptides provided. In some embodiments, the composition is a pharmaceutical composition. In some embodiments, the composition comprises a pharmaceutically acceptable carrier. In some embodiments, the composition is formulated for pulmonary delivery. In other embodiments, the aforementioned peptides (i.e., the peptides listed in Table 1) may comprise, consist essentially of, or consists of an amino acid sequence of at least 95%, 90%, 85%, 83%, 80%, 75%, or 70% identity to the peptides provided.

Referring to Table 1, Nle refers to Norleucine, an amino acid with the formula CH₃(CH₂)₃CH(NH₂)CO₂H (or the systematic name is 2-aminohexanoic acid) and Pego3 refers to a PEGylation which is a biochemical modification process of bioactive molecules with polyethylene glycol (PEG), which lends several desirable properties to proteins/peptides, antibodies, and vesicles considered to be used for therapy or genetic modification of cells. PEGylation is routinely achieved by the incubation of a reactive derivative of PEG with the target molecule. The covalent attachment of PEG to a drug or therapeutic protein can “mask” the agent from the host's immune system (reducing immunogenicity and antigenicity) and increase its hydrodynamic size (size in solution), which prolongs its circulatory time by reducing renal clearance. PEGylation can also provide water solubility to hydrophobic drugs and proteins. Additionally, an acetyl (Ac) modification may be used to closely match that of the native protein and to stabilize the peptidomimetic against enzymatic degradation by exopeptidases.

As used herein, the Pego3 modification may be used as a spacer between the peptide and the lipid or amidation. Additionally, the PEG used herein is about 0.7 kDa. In some embodiments, the PEG used herein is about 0.5 kDa, or about 0.4 kDa, or about 0.3 kDa, or about 0.2 kDa, or about 0.1 kDa, or about 0.5 kDa, or about 0.25 kDa.

Compound C892 (i.e., SEQ ID NO: 25) is a stable isostere analog of C867 (i.e., SEQ ID NO: 12) and both show near identical activities in all assays in which they were tested. Compounds C939 (i.e., SEQ ID NO: 23) and C940 (i.e., SEQ ID NO: 24) are an additional step beyond compound C892 (i.e., SEQ ID NO: 17) and now include PEGylation and lipidation (Hdc). Peptide lipidation has proven to be the most robust strategy for the generation of new peptide leads, as this modification increases the in vivo stability of peptides and decreases kidney clearance.

In some embodiments, the peptides described herein are modified by the addition of an amine or acid group to the C-terminal and acetylation or the addition of a histidine (H) to the N-terminal. In other embodiments, the peptides described herein are modified by the addition of an acid to the N-terminal. Non-limiting examples of acids that may be used to modify the N-terminal of the peptides described herein may include but are not limited to a hydroxyl group (—OH), carboxyl group/carboxylic acid group (COOH), or a combination thereof. In further embodiments, the peptides described herein are modified with a lipid group and the C-terminal and/or N-terminal.

In some embodiments, the cell is a lung cell. In other embodiments, the cell is a nasal cell. In some embodiments, the composition is for pulmonary delivery. In some embodiments, the present invention features a system comprising a pharmaceutical composition as described herein and a device for pulmonary delivery of said composition. In some embodiments, said device is a metered dose inhaler or a nebulizer. In some embodiments, the compositions described herein are for treating or preventing asthma, COPD, ARDS, or COVID-19.

The present invention features a method comprising delivering a composition comprising a peptide as described herein to a cell (e.g., a lung cell or a nasal cell) in a subject. In some embodiments, the peptide comprises an amino acid sequence of KEQCVE (SEQ ID NO: 9). In other embodiments, the peptide comprises an amino acid sequence of KEQCVE(Xaa)n (SEQ ID NO: 10). In further embodiments, the peptide comprises an amino acid sequence of (Xaa)nKEQCVE(Xaa)n (SEQ ID NO: 20). In some embodiments, peptide comprises an amino acid sequence of Ac-WGKEQCVE(Nle)(Pego3)-Hdc (SEQ ID NO: 24). Delivering the peptide to the cell (e.g., the lung cell or a nasal cell) in the subject may result in enhancing (e.g., increasing) SP-A activity in the cell and/or treating or preventing asthma, COPD, or COVID-19 in the subject (i.e., patient) or participant. In some embodiments, the composition is delivered to the cell (e.g., the lung cell) via a metered inhaler or a nebulizer.

In some embodiments, the compositions described herein reduce mucin production and/or reduce eosinophilia in said cell or lung tissue. In some embodiments, said subject is obese. In some embodiments, said peptide binds to a receptor, such as FC (CD16/32), Sirp-alpha, TLR-2, or EGFR.

The present invention further provides variants and mimetics of the SP-A peptides described herein. In some embodiments, an SP-A peptide comprises conservative, semi-conservative, and/or non-conservative substitutions relative to the peptides described herein (e.g., at positions involved in SP-A signaling or positions not involved in SP-A signaling).

Embodiments are not limited to specific substitutions. In some embodiments, the peptides described herein are further modified (e.g., substitution, deletion, or addition of standard amino acids; chemical modification, etc.). Modifications that are understood in the field include N-terminal modification, C-terminal modification (which protects the peptide from proteolytic degradation), alkylation of amide groups, hydrocarbon “stapling” (e.g., to stabilize alpha-helix conformations). In some embodiments, the peptides described herein may be modified by conservative residue substitutions, for example, of the charged residues (K to R, R to K, D to E, and E to D). In some embodiments, such conservative substitutions provide subtle changes, for example, to the receptor binding sites with the goal of improving specificity and/or biological activity. Modifications of the terminal carboxy group include, without limitation, the amide, lower alkyl amide, constrained alkyls (e.g., branched, cyclic, fused, adamantyl) alkyl, dialkyl amide, and lower alkyl ester modifications. Lower alkyl is C1-C4 alkyl. Furthermore, one or more side groups, or terminal groups, may be protected by protective groups known to the ordinarily skilled peptide chemist. The α-carbon of an amino acid may be mono- or dimethylated.

In some embodiments, one or more intra-peptide disulfide bonds are introduced (e.g., between two cysteines within the peptide. The presence of an intra-peptide disulfide bond may stabilize the peptide.

Any embodiments described herein may comprise peptidomimetics corresponding to the peptides described herein with various modifications that are understood in the field. In some embodiments, residues in the peptide sequences described herein may be substituted with amino acids having similar characteristics (e.g., hydrophobic to hydrophobic, neutral to neutral, etc.) or having other desired characteristics (e.g., more acidic, more hydrophobic, less bulky, more bulky, etc.). In some embodiments, non-natural amino acids (or naturally occurring amino acids other than the standard 20 amino acids) are substituted in order to achieve the desired properties.

In some embodiments, residues having a side chain that is positively charged under physiological conditions, or residues where a positively-charged side chain is desired, are substituted with a residue including, but not limited to: lysine, homolysine, δ-hydroxylysine, homoarginine, 2,4-diaminobutyric acid, 3-homoarginine, D-arginine, arginal (—COOH in arginine is replaced by —CHO), 2-amino-3-guanidinopropionic acid, nitroarginine (N(G)-nitroarginine), nitrosoarginine (N(G)-nitrosoarginine), methylarginine (N-methylarginine), ε-N-methyllysine, allo-hydroxylysine, 2,3-diaminopropionic acid, 2,2′-diaminopimelic acid, ornithine, sym-dimethylarginine, asym-dimethylarginine, 2,6-diaminohexinic acid, p-aminobenzoic acid and 3-aminotyrosine and, histidine, 1-methylhistidine, and 3-methylhistidine.

A neutral residue is a residue having a side chain that is uncharged under physiological conditions. A polar residue preferably has at least one polar group in the side chain. In some embodiments, polar groups are selected from hydroxyl, sulfhydryl, amine, amide and ester groups or other groups which permit the formation of hydrogen bridges. In some embodiments, residues having a side chain that is neutral/polar under physiological conditions, or residues where a neutral side chain is desired, are substituted with a residue including, but not limited to: asparagine, cysteine, glutamine, serine, threonine, tyrosine, citrulline, N-methylserine, homoserine, allo-threonine and 3,5-dinitro-tyrosine, and 3-homoserine.

Residues having a non-polar, hydrophobic side chain are residues that are uncharged under physiological conditions, preferably with a hydropathy index above 0, particularly above 3. In some embodiments, non-polar, hydrophobic side chains are selected from alkyl, alkylene, alkoxy, alkenoxy, alkylsulfanyl and alkenylsulfanyl residues having from 1 to 10, preferably from 2 to 6, carbon atoms, or aryl residues having from 5 to 12 carbon atoms. In some embodiments, residues having a non-polar, hydrophobic side chain are, or residues where a non-polar, hydrophobic side chain is desired, are substituted with a residue including, but not limited to: leucine, isoleucine, valine, methionine, alanine, phenylalanine, N-methylleucine, tert-butylglycine, octylglycine, cyclohexylalanine, β-alanine, 1-aminocyclohexylcarboxylic acid, N-methylisoleucine, norleucine, norvaline, and N-methylvaline.

In some embodiments, peptides and polypeptides are isolated and/or purified (or substantially isolated and/or substantially purified). Accordingly, in such embodiments, peptides and/or polypeptides are provided in substantially isolated form. In some embodiments, peptides and/or polypeptides are isolated from other peptides and/or polypeptides as a result of solid phase peptide synthesis, for example. Alternatively, peptides and/or polypeptides can be substantially isolated from other proteins after cell lysis from recombinant production. Standard methods of protein purification (e.g., HPLC) can be employed to substantially purify peptides and/or polypeptides. In some embodiments, the present invention provides the preparation of peptides and/or polypeptides in a number of formulations, depending on the desired use. For example, where the polypeptide is substantially isolated (or even nearly completely isolated from other proteins), it can be formulated in a suitable medium solution for storage (e.g., under refrigerated conditions or under frozen conditions). Such preparations may contain protective agents, such as buffers, preservatives, cryoprotectants (e.g., sugars such as trehalose), etc. The form of such preparations can be solutions, gels, etc. In some embodiments, peptides and/or polypeptides are prepared in lyophilized form. Moreover, such preparations can include other desired agents, such as small molecules or other peptides, polypeptides, or proteins. Indeed, such a preparation comprising a mixture of different embodiments of the peptides and/or polypeptides described here may be provided.

In some embodiments, provided herein are peptidomimetic versions of the peptide sequences described herein or variants thereof. In some embodiments, a peptidomimetic is characterized by an entity that retains the polarity (or non-polarity, hydrophobicity, etc.), three-dimensional size, and functionality (bioactivity) of its peptide equivalent but wherein all or a portion of the peptide bonds have been replaced (e.g., by more stable linkages). In some embodiments, ‘stable’ refers to being more resistant to chemical degradation or enzymatic degradation by hydrolytic enzymes. In some embodiments, the bond which replaces the amide bond (e.g., amide bond surrogate) conserves some properties of the amide bond (e.g., conformation, steric bulk, electrostatic character, capacity for hydrogen bonding, etc.). Chapter 14 of “Drug Design and Development,” Krogsgaard, Larsen, Liljefors, and Madsen (Eds) 1996, Horwood Acad. Publishers provide a general discussion of techniques for the design and synthesis of peptidomimetics and are herein incorporated by reference in its entirety. Suitable amide bond surrogates include, but are not limited to: N-alkylation (Schmidt, R. et al., Int. J. Peptide Protein Res., 1995, 46,47; herein incorporated by reference in its entirety), retro-inverse amide (Chorev, M. and Goodman, M., Acc. Chem. Res, 1993, 26, 266; herein incorporated by reference in its entirety), thioamide (Sherman D. B. and Spatola, A. F. J. Am. Chem. Soc., 1990, 112, 433; herein incorporated by reference in its entirety), thioester, phosphonate, ketomethylene (Hoffman, R. V. and Kim, H. O. J. Org. Chem., 1995, 60, 5107; herein incorporated by reference in its entirety), hydroxymethylene, fluorovinyl (Allmendinger, T. et al., Tetrahydron Lett., 1990, 31, 7297; herein incorporated by reference in its entirety), vinyl, methyleneamino (Sasaki, Y and Abe, J. Chem. Pharm. Bull. 1997 45, 13; herein incorporated by reference in its entirety), methylenethio (Spatola, A. F., Methods Neurosci, 1993, 13, 19; herein incorporated by reference in its entirety), alkane (Lavielle, S. et. al., Int. J. Peptide Protein Res., 1993, 42, 270; herein incorporated by reference in its entirety) and sulfonamido (Luisi, G. et al. Tetrahedron Lett. 1993, 34, 2391; herein incorporated by reference in its entirety).

As well as the replacement of amide bonds, peptidomimetics may involve the replacement of larger structural moieties with di- or tripeptidomimetic structures, and in this case, mimetic moieties involving the peptide bond, such as azole-derived mimetics may be used as dipeptide replacements. Suitable peptidomimetics include reduced peptides where the amide bond has been reduced to a methylene amine by treatment with a reducing agent (e.g., borane or a hydride reagent such as lithium aluminum-hydride); such a reduction has the added advantage of increasing the overall cationicity of the molecule.

The peptides and the polypeptides encompassing a substantially alpha-helical peptide region that are disclosed herein may be further derivatized by chemical alterations, such as amidation, glycosylation, acylation, sulfation, phosphorylation, acetylation, and cyclization. Such chemical alterations can be imparted through chemical or biochemical methodologies, as well as through in vivo processes, or any combination thereof.

Other peptidomimetics include peptoids formed, for example, by the stepwise synthesis of amide-functionalized polyglycines. Some peptidomimetic backbones will be readily available from their peptide precursors, such as peptides that have been permethylated; suitable methods are described by Ostresh, J. M. et al. in Proc. Natl. Acad. Sci. USA (1994) 91, 11138-11142; herein incorporated by reference in its entirety.

The peptides and polypeptides described herein may be prepared as salts with various inorganic and organic acids and bases. Such salts include salts prepared with organic and inorganic acids, for example, with HCl, HBr, H2SO4, H3PO4, trifluoroacetic acid, acetic acid, formic acid, methanesulfonic acid, toluenesulfonic acid, maleic acid, fumaric acid and camphorsulfonic acid. Salts prepared with bases include ammonium salts, alkali metal salts, e.g. sodium and potassium salts, alkali earth salts, e.g. calcium and magnesium salts, and zinc salts. The salts may be formed by conventional means, such as by reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

The peptides and polypeptides described herein can be formulated as pharmaceutically acceptable salts and/or complexes thereof. Pharmaceutically acceptable salts include acid addition salts such as those containing sulfate, hydrochloride, phosphate, sulfamate, acetate, citrate, lactate, tartrate, succinate, oxalate, methanesulfonate, ethanesulfonate, benzenesulfonate, p-toluenesulfonate, cyclohexylsulfamate and quinate. Pharmaceutically acceptable salts can be obtained from acids such as hydrochloric acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, and quinic acid. Such salts may be prepared by, for example, reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

The peptides and polypeptides described herein may be formulated as pharmaceutical compositions for use in conjunction with the methods of the present disclosure. Compositions disclosed herein may conveniently be provided in the form of formulations suitable for parenteral administration, including subcutaneous, intramuscular, and intravenous administration, nasal administration, pulmonary administration, or oral administration. Suitable formulation of peptides and polypeptides for each such route of administration is described in standard formulation treatises, e.g., Remington's Pharmaceutical Sciences by E. W. Martin. See also Wang, Y. J. and Hanson, M. A. “Parenteral Formulations of Proteins and Peptides: Stability and Stabilizers,” Journal of Parenteral Science and Technology, Technical Report No. 10, Supp. 42:2S (1988).

A pharmaceutical composition may be administered in the form that is formulated with a pharmaceutically acceptable carrier and optional excipients, adjuvants, etc., in accordance with good pharmaceutical practice. The peptide-based pharmaceutical composition may be in the form of a solid, semi-solid, or liquid dosage form: such as powder, solution, elixir, syrup, suspension, cream, drops, paste, and spray. As those skilled in the art would recognize, depending on the chosen route of administration (e.g., pill, injection, etc.), the composition form is determined. In general, it is preferred to use a unit dosage form in order to achieve an easy and accurate administration of the active pharmaceutical peptide or polypeptide. In general, the therapeutically effective pharmaceutical compound is present in such a dosage form at a concentration level ranging from about 0.5% to about 99% by weight of the total composition, e.g., in an amount sufficient to provide the desired unit dose. In some embodiments, the pharmaceutical composition may be administered in single or multiple doses. The particular route of administration and the dosage regimen will be determined by one of skill in keeping with the condition of the individual to be treated and said individual's response to the treatment. In some embodiments, a peptides-based pharmaceutical composition is provided in a unit dosage form for administration to a subject, comprising a peptide or polypeptide and one or more nontoxic pharmaceutically acceptable carriers, adjuvants, or vehicles. The amount of the active ingredient that may be combined with such materials to produce a single dosage form will vary depending upon various factors, as indicated above. A variety of materials can be used as carriers, adjuvants, and vehicles in the composition of the invention, as available in the pharmaceutical art. Injectable preparations, such as oleaginous solutions, suspensions or emulsions, may be formulated as known in the art, using suitable dispersing or wetting agents and suspending agents, as needed. The sterile injectable preparation may employ a nontoxic parenterally acceptable diluent or solvent such as sterile nonpyrogenic water or 1,3-butanediol. Among the other acceptable vehicles and solvents that may be employed are 5% dextrose injection, Ringer's injection, and isotonic sodium chloride injection (as described in the USP/NF). In addition, sterile, fixed oils may be conventionally employed as solvents or suspending media. For this purpose, any bland fixed oil may be used, including synthetic mono-, di- or triglycerides. Fatty acids such as oleic acid can also be used in the preparation of injectable compositions.

Certain of the peptides and polypeptides described herein may be substantially insoluble in water and sparingly soluble in most pharmaceutically acceptable protic solvents and in vegetable oils. In certain embodiments, cyclodextrins may be added as aqueous solubility enhancers. Cyclodextrins include methyl, dimethyl, hydroxypropyl, hydroxyethyl, glucosyl, maltosyl, and maltotriosyl derivatives of alpha-, beta-, and gamma-cyclodextrin. An exemplary cyclodextrin solubility enhancer is hydroxypropyl-beta-cyclodextrin (HPBCD), which may be added to any of the above-described compositions to further improve the aqueous solubility characteristics of the peptides or polypeptides. In one embodiment, the composition comprises 0.1% to 20% HPBCD, 1% to 15% HPBCD, or from 2.5% to 10% HPBCD. The amount of solubility enhancer employed will depend on the amount of peptide or polypeptide of the present disclosure in the composition. In certain embodiments, the peptides may be formulated in non-aqueous polar aprotic solvents such as DMSO, dimethylformamide (DMF) or N-methylpyrrolidone (NMP).

In some cases, it will be convenient to provide the peptide or polypeptide and another active agent in a single composition or solution for administration together. In other cases, it may be more advantageous to administer the additional agent separately from said polypeptide. For use, pharmaceutical compositions of the peptides and polypeptides described herein may be provided in unit dosage form containing an amount of the peptide or polypeptide effective for a single administration. Unit dosage forms useful for subcutaneous administration include prefilled syringes and injectors.

In certain embodiments, the polypeptide is administered in an amount, expressed as a daily equivalent dose regardless of dosing frequency, of 50 micrograms (“mcg”) per day, 60 mcg per day, 70 mcg per day, 75 mcg per day, 100 mcg per day, 150 mcg per day, 200 mcg per day, or 250 mcg per day. In some embodiments, the polypeptide is administered in an amount of 500 mcg per day, 750 mcg per day, or 1 milligram (“mg”) per day. In yet further embodiments, the polypeptide is administered in an amount, expressed as a daily equivalent dose regardless of dosing frequency, of 1-10 mg per day, including 1 mg per day, 1.5 mg per day, 1.75 mg per day, 2 mg per day, 2.5 mg per day, 3 mg per day, 3.5 mg per day, 4 mg per day, 4.5 mg per day, 5 mg per day, 5.5 mg per day, 6 mg per day, 6.5 mg per day, 7 mg per day, 7.5 mg per day, 8 mg per day, 8.5 mg per day, 9 mg per day, 9.5 mg per day, or 10 mg per day. In various embodiments, the polypeptide is administered on a monthly dosage schedule. In other embodiments, the polypeptide is administered biweekly. In yet other embodiments, the polypeptide is administered weekly. In certain embodiments, the polypeptide is administered daily (“QD”). In select embodiments, the polypeptide is administered twice a day (“BID”). In typical embodiments, the polypeptide is administered for at least 3 months, at least 6 months, at least 12 months, or more. In some embodiments, the polypeptide is administered for at least 18 months, 2 years, 3 years, or more.

Any carrier which can supply an active peptide or polypeptide (e.g., without destroying the peptide or polypeptide within the carrier) is a suitable carrier, and such carriers are well known in the art. In some embodiments, compositions are formulated for administration by any suitable route, including but not limited to, orally (e.g., such as in the form of tablets, capsules, granules or powders), sublingually, buccally, parenterally (such as by subcutaneous, intravenous, intramuscular, intradermal, or intrasternal injection or infusion (e.g., as sterile injectable aqueous or non-aqueous solutions or suspensions, etc.)), nasally (including administration to the nasal membranes, such as by inhalation spray), topically (such as in the form of a cream or ointment), transdermally (such as by transdermal patch), rectally (such as in the form of suppositories), etc.

In one embodiment, the pharmaceutical compositions of this invention are suitable for inhaled administration. Suitable pharmaceutical compositions for inhaled administration will typically be in the form of an aerosol or a powder. Such compositions are generally administered using well-known delivery devices, such as a nebulizer inhaler, a metered-dose inhaler (MDI), a dry powder inhaler (DPI) or a similar delivery device.

In a specific embodiment of this invention, the pharmaceutical composition comprising the active agent is administered by inhalation using a nebulizer inhaler. Such nebulizer devices typically produce a stream of high velocity air that causes the pharmaceutical composition comprising the active agent to spray as a mist that is carried into the patient's respiratory tract. Accordingly, when formulated for use in a nebulizer inhaler, the active agent is typically dissolved in a suitable carrier to form a solution. Alternatively, the active agent can be micronized and combined with a suitable carrier to form a suspension of micronized particles of respirable size, where micronized is typically defined as having about 90% or more of the particles with a diameter of less than about 10.mu.m. Suitable nebulizer devices are provided commercially, for example, by PARI GmbH (Starnberg, German). Other nebulizer devices include Respimat (Boehringer Ingelheim) and those disclosed, for example, in U.S. Pat. No. 6,123,068 to Lloyd et al. and WO 97/12687 (Eicher et al.). A representative pharmaceutical composition for use in a nebulizer inhaler comprises an isotonic aqueous solution comprising a SP-A peptide or a pharmaceutically acceptable salt or solvate or stereoisomer thereof.

In another specific embodiment of this invention, the pharmaceutical composition comprising the active agent is administered by inhalation using a dry powder inhaler. Such dry powder inhalers typically administer the active agent as a free-flowing powder that is dispersed in a patient's air-stream during inspiration. In order to achieve a free flowing powder, the active agent is typically formulated with a suitable excipient such as lactose or starch. A representative pharmaceutical composition for use in a dry powder inhaler comprises dry lactose having a particle size between about 1 μm and about 100 μm and micronized particles of SP-A peptide, or a pharmaceutically acceptable salt or solvate or stereoisomer thereof.

Such a dry powder formulation can be made, for example, by combining the lactose with the active agent and then dry blending the components. Alternatively, if desired, the active agent can be formulated without an excipient. The pharmaceutical composition is then typically loaded into a dry powder dispenser, or into inhalation cartridges or capsules for use with a dry powder delivery device. Examples of dry powder inhaler delivery devices include Diskhaler (GlaxoSmithKline, Research Triangle Park, N.C.) (see, e.g., U.S. Pat. No. 5,035,237 to Newell et al.); Diskus (GlaxoSmithKline) (see, e.g., U.S. Pat. No. 6,378,519 to Davies et al.); Turbuhaler (AstraZeneca, Wilmington, Del.) (see, e.g., U.S. Pat. No. 4,524,769 to Wetterlin); Rotahaler (GlaxoSmithKline) (see, e.g., U.S. Pat. No. 4,353,365 to Hallworth et al.) and Handihaler (Boehringer Ingelheim). Further examples of suitable DPI devices are described in U.S. Pat. No. 5,415,162 to Casper et al., U.S. Pat. No. 5,239,993 to Evans, and U.S. Pat. No. 5,715,810 to Armstrong et al., and references cited therein.

In yet another specific embodiment of this invention, the pharmaceutical composition comprising the active agent is administered by inhalation using a metered-dose inhaler. Such metered-dose inhalers typically discharge a measured amount of the active agent or a pharmaceutically acceptable salt or solvate or stereoisomer thereof using compressed propellant gas. Accordingly, pharmaceutical compositions administered using a metered-dose inhaler typically comprise a solution or suspension of the active agent in a liquefied propellant. Any suitable liquefied propellant may be employed including chlorofluorocarbons, such as CCl.sub.3F, and hydrofluoroalkanes (HFAs), such as 1,1,1,2-tetrafluoroethane (HFA 134a) and 1,1,1,2,3,3,3-heptafluoro-n-propane, (HFA 227). Due to concerns about chlorofluorocarbons affecting the ozone layer, formulations containing HFAs are generally preferred. Additional optional components of HFA formulations include co-solvents, such as ethanol or pentane, and surfactants, such as sorbitan trioleate, oleic acid, lecithin, and glycerin. See, for example, U.S. Pat. No. 5,225,183 to Purewal et al., EP 0717987 A2 (Minnesota Mining and Manufacturing Company), and WO 92/22286 (Minnesota Mining and Manufacturing Company). A representative pharmaceutical composition for use in a metered-dose inhaler comprises from about 0.01% to about 5% by weight of a compound of SP-A peptide or a pharmaceutically acceptable salt or solvate or stereoisomer thereof; from about 0% to about 20% by weight ethanol; and from about 0% to about 5% by weight surfactant; with the remainder being an HFA propellant.

Such compositions are typically prepared by adding chilled or pressurized hydrofluoroalkane to a suitable container containing the active agent, ethanol (if present) and the surfactant (if present). To prepare a suspension, the active agent is micronized and then combined with the propellant. The formulation is then loaded into an aerosol canister, which forms a portion of a metered-dose inhaler device. Examples of metered-dose inhaler devices developed specifically for use with HFA propellants are provided in U.S. Pat. No. 6,006,745 to Marecki and U.S. Pat. No. 6,143,277 to Ashurst et al. Alternatively, a suspension formulation can be prepared by spray drying a coating of surfactant on micronized particles of the active agent. See, for example, WO 99/53901 (Glaxo Group Ltd.) and WO 00/61108 (Glaxo Group Ltd.), the disclosures of which are incorporated in their entirety herein by reference.

For additional examples of processes of preparing respirable particles, and formulations and devices suitable for inhalation dosing see U.S. Pat. No. 6,268,533 to Gao et al., U.S. Pat. No. 5,983,956 to Trofast, U.S. Pat. No. 5,874,063 to Briggner et al., and U.S. Pat. No. 6,221,398 to Jakupovic et al.; and WO 99/55319 (Glaxo Group Ltd.) and WO 00/30614 (AstraZeneca AB), the disclosures of which are incorporated in their entirety herein by reference.

In some embodiments, peptides/polypeptides are provided in pharmaceutical compositions and/or co-administered (concurrently or in series) with one or more additional therapeutic agents. Such additional agents may be for the treatment or prevention of lung inflammation (e.g., asthma). Additional agents may include, but are not limited to: short-acting beta2-adrenoceptor agonists (SABA), such as salbutamol (albuterol USAN); long acting beta agonist (LABA) such as salmeterol and formoterol anticholinergic medications, such as ipratropium bromide, inhaled epinephrine, inhaled corticosteroids such as budesonide, fluticasone, mometasone or ciclesonide, systemic corticosteroids such as prednisone or methylprednisolone; leukotriene receptor antagonists (e.g., montelukast and zafirlukast); or combinations thereof.

Without wishing to limit the present invention to any theories of mechanisms, it is believed that the purified peptides described herein co-administered (concurrently or in series) with one or more additional therapeutic agents (e.g., steroids) allows for a lesser dose of the steroid to treat severe asthma. Thus, combination therapy may have a steroid sparing effect given different mechanisms of action as compared to inhaled corticosteroids alone or with combination therapy (inhaled corticosteroids (ICS)/LABA).

Non-limiting examples of combination therapies include purified peptides as described herein and ICS; purified peptides as described herein and ICS/LABA; purified peptides as described herein and ICS/LABA/LAMA (long-acting muscarinic antagonist); purified peptides as described herein and SABA; or purified peptides as described herein and ICS/SABA.

The aforementioned combination therapies may be used to treat any inflammatory lung disease (e.g., asthma, COPD, or COVID). In some embodiments, the inflammatory lung disease is treated with a purified peptide as described herein (e.g., an inhaled purified peptide). In other embodiments, the inflammatory lung disease is treated with a purified peptide as described herein (e.g., an inhale purified peptide) and a steroid.

In some embodiments, provided herein are methods for treating patients suffering from (or at risk of) lung disease (e.g., asthma) and/or in need of treatment (or preventative therapy). In some embodiments, patients are obese or are not obese can benefit. In some embodiments, subjects are identified as having an SP-A genotype associated with an increased risk of asthma or severe asthma (e.g., those genotypes described herein).

In some embodiments, a pharmaceutical composition comprising at least one SP-A peptide or polypeptide described herein is delivered to such a patient in an amount and at a location sufficient to treat the condition. In some embodiments, peptides and/or polypeptides (or pharmaceutical composition comprising such) can be delivered to the patient systemically or locally, and it will be within the ordinary skill of the medical professional treating such patient to ascertain the most appropriate delivery route, time course, and dosage for treatment. It will be appreciated that application methods of treating a patient most preferably substantially alleviate or even eliminate such symptoms; however, as with many medical treatments, application of the inventive method is deemed successful if, during, following, or otherwise as a result of the inventive method, the symptoms of the disease or disorder in the patient subside to an ascertainable degree.

The present disclosure is not limited to the treatment of asthma. Any inflammatory conditions known in the art or otherwise contemplated herein may be treated in accordance with the presently disclosed and claimed inventive concept(s). Non-limiting examples of disease conditions having inflammation associated therewith include infection-related or non-infectious inflammatory conditions in the lung (e.g., asthma, sepsis, chronic obstructive pulmonary disease (COPD), COVID-19, lung infections, Respiratory Distress Syndrome, bronchopulmonary dysplasia, etc.); infection-related or non-infectious inflammatory conditions in other organs (e.g., colitis, Inflammatory Bowel Disease, diabetic nephropathy, hemorrhagic shock); inflammation-induced cancer (i.e., cancer progression in patients with colitis or Inflammatory Bowel Disease); and the like.

### EXAMPLES

The following is a non-limiting example of the present invention. It is to be understood that said example is not intended to limit the present invention in any way. Equivalents or substitutes are within the scope of the present invention.

Example 1: Example 1 demonstrates the genotype of SP-A at position 223 affects lung function and asthma control. Previous studies have shown that SP-A derived from asthmatic subjects is dysfunctional in regulating inflammatory conditions, such as IL-8 and MUCSAC production. To determine if the genotype of SP-A influences lung function, lung physiology and asthma control, SP-A was genotyped and measured in 53 mild-moderate asthmatics not on controller therapy. FIG. 15A shows of the 53 asthmatics screened, those that are homozygous for the minor allele (SP-A2 K223K) have lower lung function (FEV1%) than those subjects with the Q223Q or Q223K genotype. Moreover, this cohort of asthmatic subjects demonstrates worse asthma control (asthma control questionnaire; ACQ) than asthmatics with the AC or CC genotype (FIG. 15B).

Additionally, SP-A humanized mice exhibit different phenotypes in an allergic model. In order to study more mechanistically the effect of genetic variation in SP-A at position 223Q/K in asthma, SP-A humanized mice that express either the SP-A223Q (major allele) or SP-A223K (minor allele) were generated. It was discovered that when challenged in the ova model of allergic airways disease the SP-A223Q allele confers more protection as compared to the SP-A223K allele. FIG. 15A shows SP-A223Q mice have significantly decreased eosinophilia as compared to mice deficient in SP-A 24 hrs post challenge and they also have significantly reduced mucin production (PAS scores) 7 days post challenge (FIG. 15B).

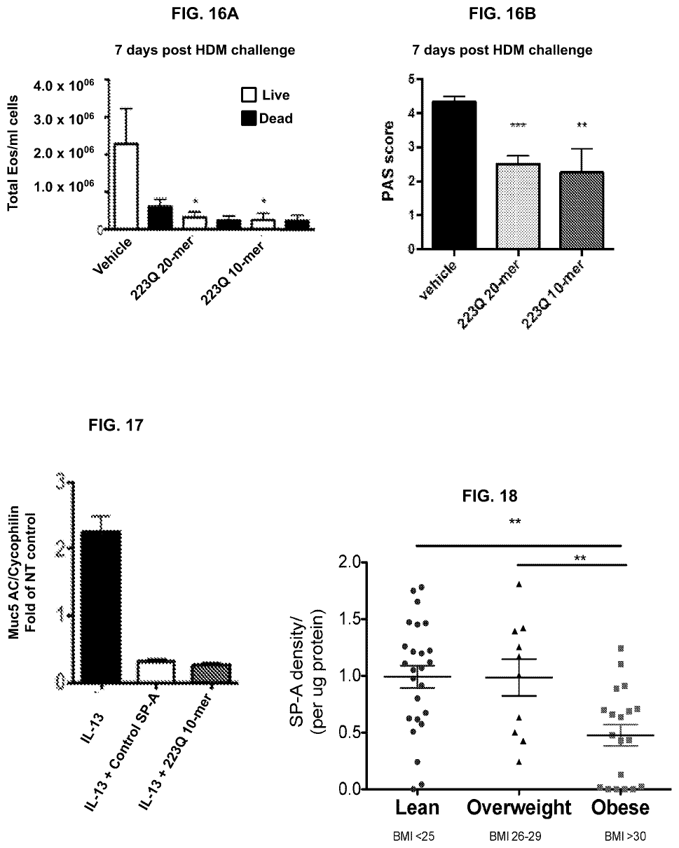

SP-A peptides that encompass the 223Q active site attenuate airway eosinophils and mucin production in an allergic model. Since asthmatic subjects harboring the 223K (minor) allele had worse asthma control and lung function as compared to individuals with the 223Q (major) allele and since similar findings were observed in the SP-A humanized mice in an allergic model, experiments were performed to find the active region of SP-A. It was determined that the active peptide is a 10 AA peptide that includes the 223Q site and is located in the carbohydrate recognition domain of endogenous SP-A. As shown in FIGS. 16A and 16B, SP-A deficient mice were challenged in the HDM model and 24 hrs after the last challenge, either a 20AA SP-A (PAGRGKEQCVEMYTDGQWND (SEQ ID NO: 8) or a 10AA SP-A (KEQCVEMYTD (SEQ ID NO: 4)) was given and compared to those mice receiving vehicle treatment. Those mice receiving the SP-A peptides had significantly lower eosinophilia in the lavage compartment (FIG. 16A) and less mucin production (FIG. 16B) as compared to vehicle treated mice.

SP-A peptides (10AA) that encompass the 223Q active site attenuate phenotypes in primary human airway epithelial cells from asthmatics. Experiments were conducted wherein airway epithelial cells from two subjects with asthma not on controller therapy were cultured at air liquid interface for two weeks. In separate conditions the cells were exposed to each peptide at 20 μg/ml for 30 minutes followed by IL-13 at 50 ng/ml and incubated for 48 hours. Cells were placed in Trizol MUCSAC was determined by RT-PCR. FIG. 17 shows a dramatic reduction in MUCSAC expression with each of the peptides to the level of negative control. The 10 AA length is especially useful as a therapeutic agent as its size renders it able to be packaged into an inhaler type of device for delivery to the airways. This experiment demonstrates that the 223Q peptide has efficacy in suppressing mucin gene expression in human airway epithelial cells in the setting of IL-13 exposure.

In summary, these experiments demonstrated that SP-A genotype at position 223 affects lung function and asthma control; SP-A humanized mice exhibit different phenotypes in an allergic model dependent on position 223Q/K, SP-A peptides that encompass the 223Q active site attenuate phenotypes in an allergic model, SP-A peptide (10AA) that encompasses the 223Q active site attenuates Muc5AC in primary human airway epithelial cells from asthmatics.

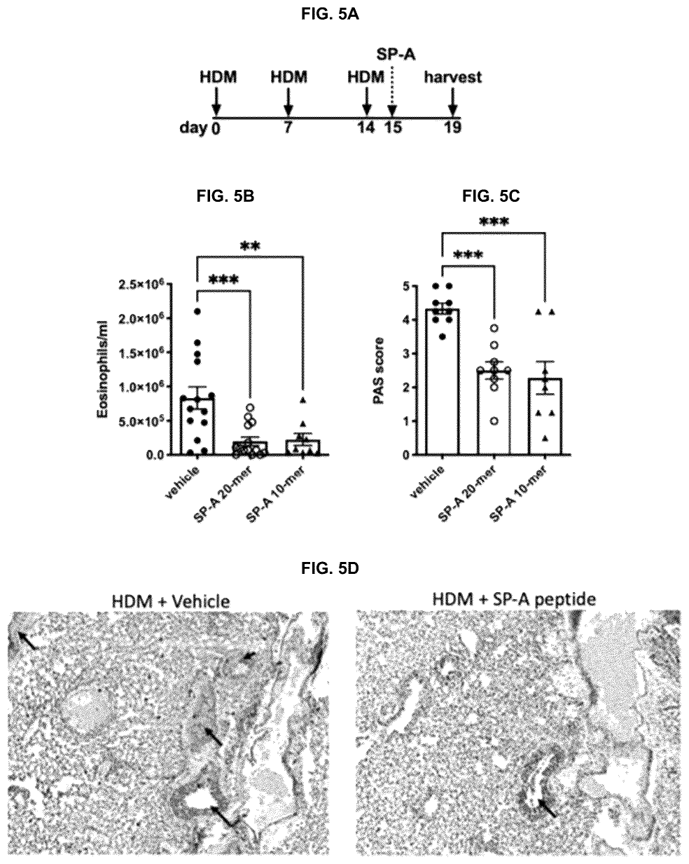

Regarding FIG. 5A-5D, WT C57BL/6 mice were sensitized and challenged in the house dust mite (HDM) model according to standard methods on days 0, 7, 14 (black arrows). Twenty-four hours after the last challenge, mice received either scrambled vehicle or a 20-mer SP-A peptide (dotted arrow) that encompassed the active site containing 223Q (at a physiologic dose of 25 μg/mouse delivered in 40 μl of sterile saline) by oropharyngeal instillation. Lung histological sections were analyzed for mucin production as assessed by PAS stain/scoring to determine if the SP-A peptide could protect from HDM-induced airway mucin production. Similar protective effects were observed on days 5 and 7 after SP-A peptide treatment. Peptide sequence: PAGRGKEQCVEMYTDGQWND (SEQ ID NO: 8).

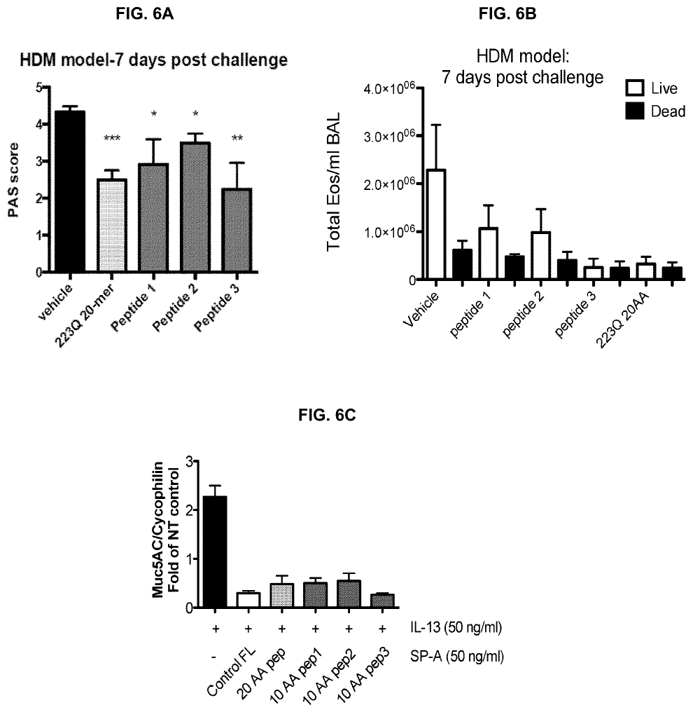

Regarding FIGS. 6A and 6B, WT C57BL/6 mice were sensitized and challenged in the house dust mite (HDM) model according to standard methods described in the aforementioned paragraph (see FIG. 5A). Twenty-four hours after the last challenge, mice received either scrambled vehicle, 20-mer, or 10-mer SP-A peptides (dotted arrow) that encompassed the active site containing 223Q (at a physiologic dose of 25 μg/mouse delivered in 40 μl of sterile saline) by oropharyngeal instillation. In FIG. 6A, lung histological sections were analyzed for mucin production as assessed by PAS stain/scoring to determine if the SP-A peptide could protect from HDM-induced airway mucin production. Similar protective effects were observed on days 5 and 7 after SP-A peptide treatment. In FIG. 6B, bronchoalveolar lavage samples were analyzed for eosinophilia to determine if the SP-A peptide could protect from HDM-induced airway eosinophilia by decreasing eosinophil viability. Viability was assessed by Trypan blue exclusion on a cell countess. Peptide sequences: 20-mer PAGRGKEQCVEMYTDGQWND (SEQ ID NO: 8), peptide 1 PAGRGKEQCV (SEQ ID NO: 2), peptide 2 EMYTDGQWND (SEQ ID NO: 3), peptide 3 KEQCVEMYTD (SEQ ID NO: 4).

Regarding FIG. 6C, human bronchial epithelial cells obtained from well-phenotyped asthmatic participants by bronchoscopy were grown at ALI for two weeks prior to experimentation. For challenge, each of the SP-A test peptides (50 μg/ml) were added to the apical compartment at least 30 minutes prior to IL-13 challenge. Muc5AC was analyzed by RT-PCR from cell lysates and analyzed as a fold over the control samples. Peptide sequences: 20-mer PAGRGKEQCVEMYTDGQWND (SEQ ID NO: 8), peptide 1 PAGRGKEQCV (SEQ ID NO: 2), peptide 2 EMYTDGQWND (SEQ ID NO: 3), peptide 3 KEQCVEMYTD (SEQ ID NO: 4) FL=full length oligomeric SP-A that is extracted from lavage of alveolar proteinosis individuals.

Regarding FIG. 18, SP-A expression was analyzed from the bronchoalveolar lavage of lean, overweight and obese individuals with and without asthma by Western blot.

FIGS. 5A-5D, 6A-6C, 15A-15B, and 18 show that SP-A223Q humanized mice have less mucus than the 223K mice in an allergic model (Ova model; FIGS. 15A-15B), that SP-A223Q humanized mice have less mucus than the 223K mice in an allergic model (HDM (house dust mite) model; FIGS. 5A-5D), that shortened 10AA peptides reduce mucin production in mouse HDM model (FIG. 6A), that shortened 10AA peptides reduce eosinophilia in mouse HDM model (FIG. 6B), that shortened 10AA peptides reduce Mucin (Muc5AC RNA) in human primary cells (FIG. 6C), and that SP-A is significantly decreased in obesity (FIG. 18).

Example 2: Example 2 describes the use of an HDM sensitization and challenge model in 10-12 adult primates that have been selected by pre-screening for baseline sensitivity to methacholine challenge to test SP-A therapeutic peptide in a crossover study design.

First, HDM allergen is administered to all 10 primates by subcutaneous injection biweekly for 10 weeks, at which time animals are tested for HDM skin reactivity. Next, HDM mask exposure is performed biweekly for a total of 8 weeks. After this HDM challenge period, airway hyperresponsiveness is assessed in all 12 primates and lavage fluid and biopsy specimens are collected for analysis.

After the first round of analysis, in which the level of response each primate has to the HDM model is assessed, the 10 primates are divided into two test groups in a randomized, double-blind crossover design: group 1 (n=6) receive the SP-A peptide followed by washout, then placebo; group 2 (n=6) receive placebo first, followed by washout and then SP-A peptide. SP-A peptide and placebo are given biweekly via intranasal administration for four weeks, while primates are still receiving HDM mask treatments bi-weekly. The dosing of SP-A and placebo is approximately 24 hrs after the HDM mask exposure.

At the end of the first study period at 4 weeks, primates are analyzed for airway hyperresponsiveness. Bronchoscopy is performed for lavage fluid and endobronchial biopsy. After a four week washout, primates are again challenged with HDM mask treatments bi-weekly during study period 2. Group 1 (n=6) receive placebo and group 2 (n=6) receive SP-A peptide biweekly following HDM mask treatment for 4 weeks as described above. After completion of study period 2, all primates are analyzed for airway hyperresponsiveness and bronchoscopy are performed for lavage fluid and endobronchial biopsy.

Statistical Analysis: Primary outcome variables include the airways hyperresponsiveness, lavage and tissue eosinophilia and tissue mucin production. These variables are analyzed by using a two-period crossover analysis of variance model. Carryover effects are tested at the 10% alpha level, whereas period and treatment effects are tested at the 5% alpha level. Data is expressed as means±SEM.

It is anticipated that the SP-A peptide are able to alleviate phenotypes associated with asthma in the HDM allergic model in primates. Thus far, it was shown that one dose of peptide in mice 24 hrs after the last HDM challenge (which is the peak of inflammation) is able to significantly reduce mucin production and eosinophilia in the lavage compartment and in the lung tissue. It is anticipated that giving peptides biweekly over the course of 4 weeks significantly reduces eosinophilia and mucin production as compared to placebo.

Example 3: Example 3 demonstrates that SP-A peptide protects against AHR in HDM (house dust mite) model. As shown in FIG. 10, WT C57BL/6 mice were sensitized and challenged in the HDM model according to standard methods (arrow). Twenty-four hours after each challenge, mice received either scrambled vehicle or a 10-mer SP-A peptide (KEQCVEMYTD (SEQ ID NO: 4)) (arrow) that encompassed the active site containing 223Q (at a physiologic dose of 25 mg/mouse delivered in 40 ml of sterile saline) by oropharyngeal instillation. Pulmonary function tests were conducted 3-5 days post HDM challenge to determine if the SP-A peptide could protect from methacholine-induced airway hyper-responsiveness (AHR). Mice that received the SP-A peptide after HDM challenge had attenuated: overall resistance (Rrs) and central airways resistance (Rn) as compared to HDM challenged mice that received vehicle treatment. Similar protective effects were observed on days 3 and 5 after SP-A peptide treatment.

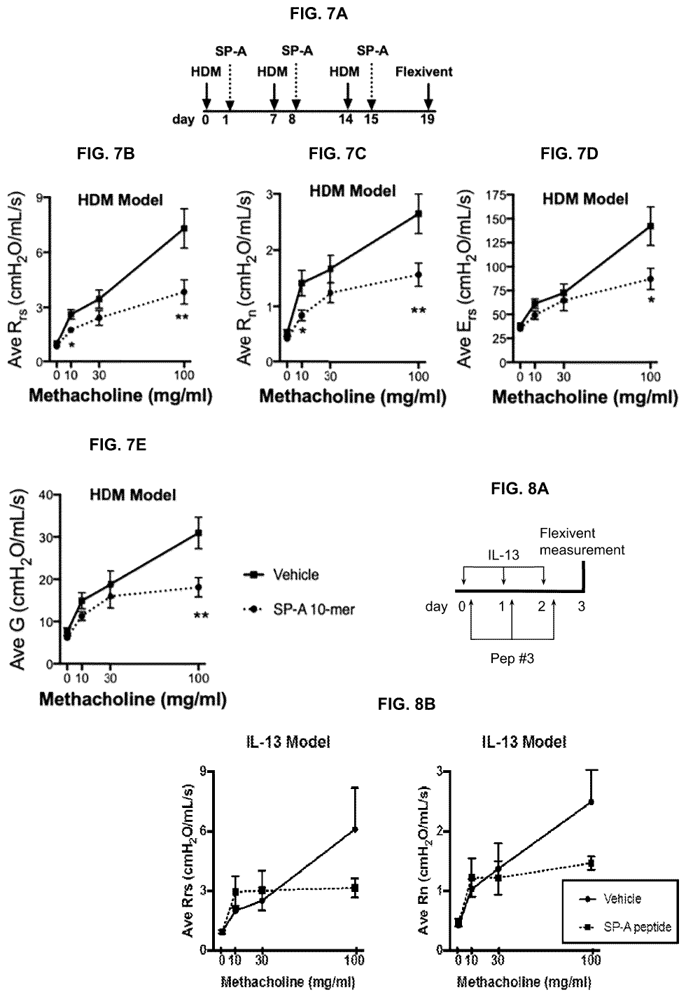

Example 4: Example 4 4 demonstrates that SP-A peptide protects against airway hyper-responsiveness (AHR) in IL-13 model. WT C57BL/6 mice were challenged with 3.9 μg of IL-13 (arrow) once a day for 3 consecutive days via oropharyngeal instillation. As shown in FIG. 7A-7E, Two hours after each challenge, mice received either scrambled vehicle or a 10-mer SP-A peptide (KEQCVEMYTD (SEQ ID NO: 4)) (arrow) that encompassed the active site containing 223Q (at a physiologic dose of 25 mg/mouse delivered in 40 ml of sterile saline) by oropharyngeal instillation. Pulmonary function tests were conducted 24 hours post IL-13 challenge to determine if the SP-A peptide could protect from methacholine-induced airway hyper-responsiveness (AHR). Mice that received the SP-A peptide after HDM challenge had attenuated: overall resistance (Rrs) and central airways resistance (Rn) as compared to HDM challenged mice that received vehicle treatment.

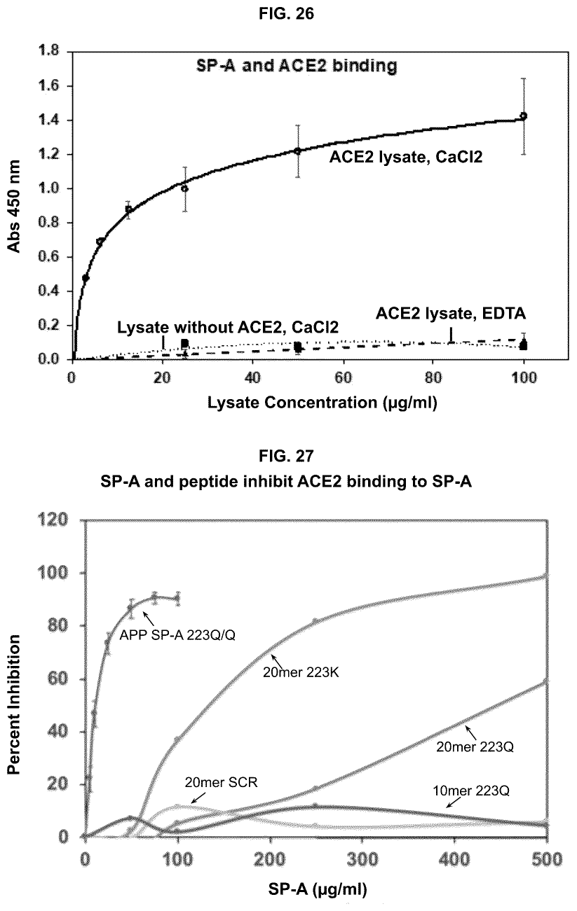

Example 5: Example 5 demonstrates that SP-A peptide protects against an SARS-COV-2 infection. As shown in FIG. 26 full-length SP-A binds to ACE2 in dose-dependent and CaCl2 dependent-manner. A 96-well plate assay was devised in which wells were coated with full-length human SP-A (500 ng/well) extracted from BAL obtained from alveolar proteinosis patients. Next, lysates from HEK293T cells transfected with ACE2-expressing plasmids were added in varying concentrations. After vigorous washings, ACE2 was detected adding an anti-human ACE2 antibody and the developing substrate. Absorbance at 450 nm wavelength was read by plate reader. Lysates over-expressing ACE2 bound to SP-A in a dose-dependent manner (FIG. 26, solid line). Since SP-A commonly binds to pathogens in a calcium-dependent manner via the carbohydrate recognition domain (CRD), it was next determined if SP-A binding to ACE2 was calcium dependent. There was no detectable SP-A binding to ACE2 when the chelator, EDTA, is present (FIG. 26, dotted line), suggesting that binding occurs through the lectin domain of SPA. No SP-A binding was detected when lysates not overexpressing ACE2 were used as negative controls, (FIG. 26, dashed line).

Referring to FIG. 27, SP-A peptide mimetics derived from CRD compete with full-length SP-A for ACE2 binding. Both full-length SPA and SP-A 20-mer peptides competed for (i.e., inhibited) ACE binding to plate-bound SP-A, while a scrambled 20-mer peptide had no effect (FIG. 27). In the general population, a specific variant at position 223 in SP-A2 is associated with lung phenotypes. In particular, SP-A containing a lysine at position 223 (223K) binds preferentially to the respiratory pathogen, Mycoplasma pneumoniae, as compared to when a glutamine is present at this position (223Q). Additionally, peptides derived from this region have activity in various models of infection and inflammation as previously described herein. For these studies, Full-length SP-A or SPA 20-mer peptides were assessed, to determine if the peptides competed for (i.e., inhibited) ACE2 binding to plate bound SP-A. While both full-length SP-A and the 223K containing 20-mer competed effectively for binding, the 20-mer scrambled (SCR) peptide had little to no effect and the 223Q containing 20-mer had moderate effect (n=3 experiments).

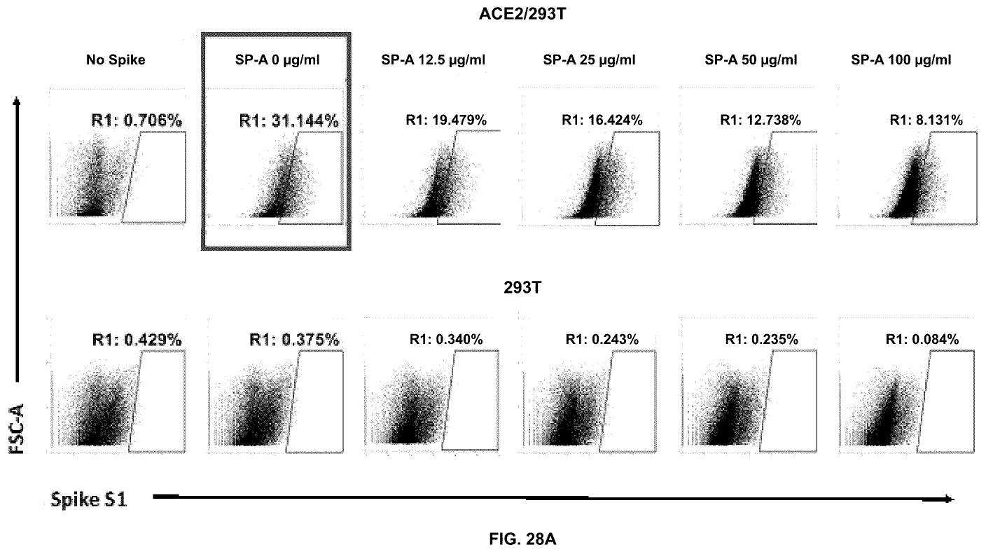

To assess the effects of SP-A on SARS-COV2 S protein attachment, an S1 protein binding assay was used. Cells were incubated with a recombinant His-tagged S1 subunit comprising the SARS-COV-2 receptor binding domain, followed by a Alexa Fluor-conjugated anti-His antibody. S1 protein binding to cells was assessed by flow cytometry. The ability of this assay to specifically detect ACE2-mediated S1 protein cellular binding was validated using HEK293T cells, that were non-transfected or stably transfected with human ACE2 (ACE2/HEK293T). Whereas S1 binding was detected in less than 1% of non-transfected HEK293T cells and ACE2/HEK293T cells without the S1 protein incubated with anti-His-AF (FIG. 28A), approximately ˜30% ACE2/HEK293T cells bound S1 protein in the absence of SP-A (FIG. 28A, box). The addition of full-length SP-A resulted in dose-dependent reduction in S1 binding, with the highest concentration of SP-A reducing binding by ˜70% (FIG. 28B).

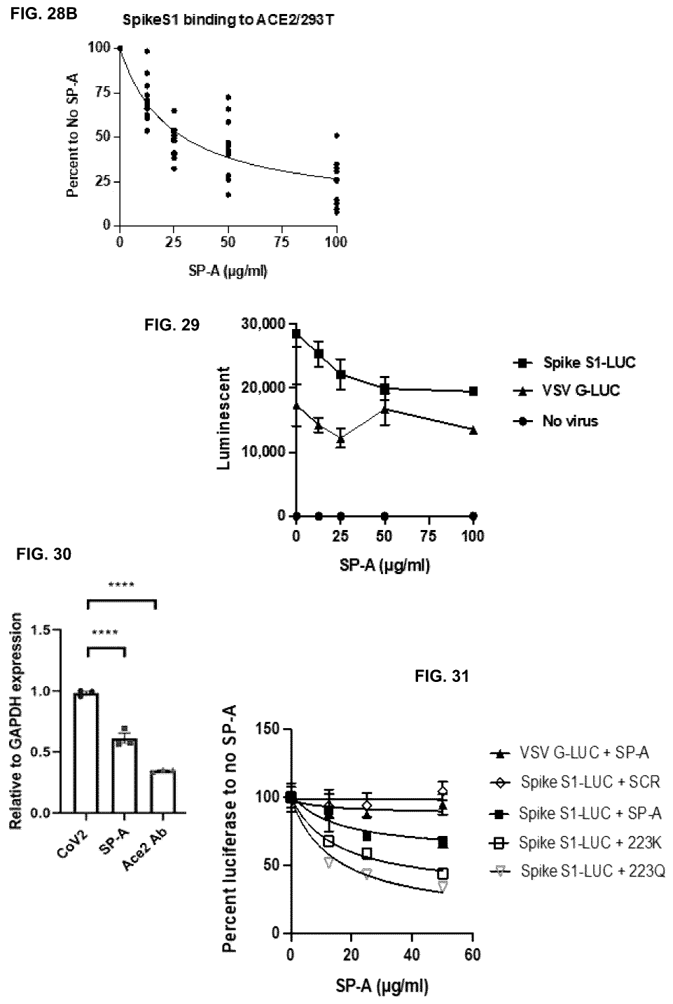

To investigate whether SP-A inhibition of and SARS-COV-2 S1 protein attachment to HEK293T ACE overexpressing cells also reduces S protein-mediated SARS-COV-2 entry into these cells, the entry of replication-deficient, SARSCOV-2 S1 protein-pseudotyped lentiviral particles into cells preincubated with SP-A or PBS were directly measured. The lentiviral particles carried a Luciferase reporter gene that was transcribed and translated by transduced cells, and were pseudotyped with SARS-COV-2 S protein or the G glycoprotein of the pantropic VSV (positive transduction control). VSV-G-pseudotyped particles and SARS-COV-2-pseudotyped particles efficiently transduced HEK293T ACE cells. While SP-A had little to no impact on VSV G-LUC transfection, SP-A dose-dependently reduced the transduction efficiency of SARSCOV-2 S protein-pseudotyped lentiviral particles (FIG. 29).

Referring now to FIG. 30, SP-A attenuates SARS-COV-2 N1 gene expression in a live infection model. Three-dimensional (3D) alveolar organoid cultures were established by resuspending fractioned HTII-280+distal epithelial cells with MRC5 human lung fibroblast cells in a 50:50 (v/v) ratio of Matrigel and Pneumacult ALI medium in 24 well format transwell inserts according to common methods. Cultures were used for SARS-Co-V2 infection after 15-20 days. Prior to infection, Matrigel was dissolved by adding 500 μL of Dispase (500 μg/ml) to the apical and basement chambers of inserts and incubating for 1 hour at 37° C. Cultures were harvested, washed with ice cold PBS and gently dispersed with a P1000 tip by pipetting up and down 3 times such that the organoids were ‘popped open’, exposing the apical surface of the cells. Organoids were treated by resuspending in 100 μL of media containing SP-A (50 μg/ml) per well. After 3 hrs of pretreatment, SARS-COV-2 inoculum (1×10\hat{ }4 TCID50 per well) was added to the cultures in a 2 ml conical tube and incubated for 2 hours at 37° C. (5% CO2). Every 15 minutes, tubes were gently mixed to facilitate virus adsorption on the cells. Subsequently, inoculum was replaced with fresh Pneumacult ALI medium and cultures were transferred to the apical chamber of the insert in 100 μL volume with 500 μL of the medium in the basement chamber. Cultures were incubated at 37° C. (5% CO2) and harvested at 2 dpi. SP-A was maintained in the media for the duration of culture post infection. Organoids without drug treatment in the presence (CoV-2) or absence (mock) of viral infections were included as controls. Viral infection/replication was assessed by performing RT-qPCR for relative Nucleoprotein (N) gene expression using the 2019-nCOV_N1 primers obtained from the Center for Disease Control resources for research labs.

FIG. 31 shows SP-A 223Q and 223K 20-mer peptides reduced transduction of S1 protein-pseudotyped lentiviral particles in vitro to cells overexpressing ACE2. Using the same system as detailed in FIG. 29 above, whether SP-A derived 20-mer peptides behaved similar to full-length SP-A in the ability to inhibit S protein-mediated SARS-COV-2 entry into these cells was next investigated. Again, the entry of replication-deficient, SARSCOV-2 S1 protein-pseudotyped lentiviral particles into cells preincubated with SP-A, SP-A 223Q and 223K peptides or PBS were directly measured. The lentiviral particles carried a Luciferase reporter gene that is transcribed and translated by transduced cells, and were pseudotyped with SARS-COV-2 S protein or the G glycoprotein of the pantropic VSV (positive transduction control). VSV-G-pseudotyped particles and SARS-COV-2-pseudotyped particles efficiently transduced HEK293T ACE cells. SP-A 223 Q and 223K peptides dose-dependently reduced the transduction efficiency of SARSCOV-2 S protein-pseudotyped lentiviral, to a greater extent than full-length SP-A at each given concentration (FIG. 31).

### Embodiments

The following embodiments are intended to be illustrative only and not to be limiting in any way.

Embodiment 1: A method of treating or preventing an inflammatory lung disease in a subject in need thereof, the method comprising administering a therapeutically effective amount of a purified peptide comprising an amino acid sequence of KEQCVE (SEQ ID NO: 9) to the subject.

Embodiment 2: The method of embodiment 1, wherein the peptide is selected from a group consisting of SEQ ID NO: 4, SEQ ID NO: 8, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, and SEQ ID NO: 24.

Embodiment 3: A method of treating or preventing an inflammatory lung disease in a subject in need thereof, the method comprising administering a therapeutically effective amount of a purified peptide comprising an amino acid sequence of Ac-WGKEQCVE(Nle)(Pego3)-Hdc (SEQ ID NO: 24) to the subject.

Embodiment 4: The method of any one of embodiments 1-3, wherein the inflammatory lung disease comprises asthma, chronic obstructive pulmonary disease (COPD), or COVID-19.

Embodiment 5: The method of embodiment 4, wherein asthma is selected from a group consisting of type 2 asthma, non-type 2 asthma, early onset asthma, late-onset asthma, obesity-associated asthma, exercise-induced asthma, asthma exacerbations, asthma associated with pollutants to cigarette smoke, or asthma associated with infections.

Embodiment 6: The method of any one of embodiments 1-5, wherein the purified peptide is in a preparation for aerosolization or subcutaneous injection.

Embodiment 7: The method of any one of embodiments 1-6, wherein the purified peptide is administered via nasal inhalation, subcutaneously, or orally.

Embodiment 8: The method of any one of embodiments 1-7, further comprising administering the purified peptide with one or more additional therapeutic agents.

Embodiment 9: The method of embodiment 8, wherein the one or more therapeutic agents are administered concurrently or in series with the purified peptide.

Embodiment 10: The method of embodiment 8 or embodiment 9, wherein the one or more therapeutic agents comprise short-acting beta2-adrenoceptor agonists (SABA), long-acting beta agonist (LABA), anticholinergic medications, leukotriene receptor antagonists, or a combination thereof.

Embodiment 11: The method of embodiment 10, wherein anticholinergic medications comprise ipratropium bromide, inhaled epinephrine, and inhaled corticosteroids.

Embodiment 12: The method of embodiment 11, wherein inhaled corticosteroids comprise budesonide, fluticasone, mometasone, or ciclesonide.

Embodiment 13: A pharmaceutical composition for enhancing (e.g., increasing) SP-A activity in a cell, comprising a purified peptide comprising an amino acid sequence of KEQCVE (SEQ ID NO: 9) in a pharmaceutical carrier.

Embodiment 14: The composition of embodiment 13, wherein the purified peptide comprises an amino acid sequence of KEQCVE(Xaa)n (SEQ ID NO: 10); wherein n ranges from 4-16 amino acids; wherein Xaa is any natural or non-natural amino acids

Embodiment 15: The composition of embodiment 13, wherein the peptide is selected from a group consisting of SEQ ID NO: 4, SEQ ID NO: 8 SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, and SEQ ID NO: 24.

Embodiment 16: A pharmaceutical composition for enhancing (e.g., increasing) SP-A activity in a cell, comprising a purified peptide comprising an amino acid sequence of Ac-WGKEQCVE(Nle)(Pego3)-Hdc (SEQ ID NO: 24) in a pharmaceutical carrier.

Embodiment 17: The composition of any one of embodiments 13-16, wherein the composition is in a preparation for aerosolization, subcutaneous injection, or pulmonary delivery.

Embodiment 18: The composition of any one of embodiments 13-17, wherein the cell is a lung cell or a nasal cell.

Embodiment 19: A system comprising the pharmaceutical composition of any one of embodiments 13-18 and a device for pulmonary delivery of said composition.

Embodiment 20: The system of embodiment 19, wherein said device is a metered dose inhaler or a nebulizer.

Embodiment 21: A method of treating an inflammatory lung disease in a subject in need thereof, the method comprising administering a therapeutically effective amount of a pharmaceutical composition according to any one of embodiments 13-18 to the subject.

Embodiment 22: The method of embodiment 21, wherein the inflammatory lung disease comprises asthma, chronic obstructive pulmonary disease (COPD), or COVID-19.

Embodiment 23: The method of embodiment 22, wherein asthma is selected from a group consisting of type 2 asthma, non-type 2 asthma, early onset asthma, late-onset asthma, obesity-associated asthma, exercise-induced asthma, or asthma exacerbations.

Embodiment 24: The method of any one of embodiments 21-23, wherein the composition is administered via nasal inhalation, subcutaneously, or orally.

Embodiment 25: The method of any one of embodiments 21-24, further comprising administering the composition with one or more additional therapeutic agents.

Embodiment 26: The method of embodiment 25, wherein the one or more therapeutic agents are administered concurrently or in series with the purified peptide.

Embodiment 27: A method comprising: delivering a composition comprising a purified peptide to a cell in a subject, said peptide comprising an amino acid sequence of KEQCVE (SEQ ID NO: 9), wherein said delivering results in enhancing (e.g., increasing) SP-A activity in the cell, treating an inflammatory lung disease in the subject, or both.

Embodiment 28: The method of embodiment 27, wherein the peptide is selected from a group consisting of SEQ ID NO: 4, SEQ ID NO: 8, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, and SEQ ID NO: 24.

Embodiment 29: A method comprising: delivering a composition comprising a purified peptide to a cell in a subject, said peptide comprising an amino acid sequence of Ac-WGKEQCVE(Nle)(Pego3)-Hdc (SEQ ID NO: 24), wherein said delivering results in enhancing (e.g., increasing) SP-A activity in the cell, treating an inflammatory lung disease in the subject, or both.

Embodiment 30: The method of any one of embodiments 27-29, wherein the inflammatory lung disease comprises asthma, chronic obstructive pulmonary disease (COPD), or COVID-19.

Embodiment 31: The method of embodiment 30, wherein asthma is selected from a group consisting of type 2 asthma, non-type 2 asthma, early onset asthma, late-onset asthma, obesity-associated asthma, exercise-induced asthma, or asthma exacerbations.

Embodiment 32: The method of any one of embodiments 27-31, wherein the purified peptide is in a preparation for aerosolization or subcutaneous injection.

Embodiment 33: The method of any one of embodiments 27-32, wherein the purified peptide is administered via nasal inhalation, subcutaneously, or orally.

Embodiment 34: The method of any one of embodiments 27-33, wherein said composition reduces mucin production and/or reduces eosinophilia in said cell.

Embodiment 35: The method of any one of embodiments 27-34, further comprising administering the composition with one or more additional therapeutic agents.

Embodiment 36: The method of embodiment 35, wherein the one or more therapeutic agents are administered concurrently or in series with the purified peptide.

Embodiment 37: The method of any one of embodiments 27-36, wherein said subject is obese.

Embodiment 38: A purified peptide for use in a method of treating an inflammatory lung disease, the purified peptide comprising an amino acid sequence of KEQCVE (SEQ ID NO: 9).

Embodiment 39: The peptide of embodiment 38, wherein the peptide is selected from a group consisting of SEQ ID NO: 4, SEQ ID NO: 8, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, and SEQ ID NO: 24.

Embodiment 40: A purified peptide for use in a method of treating an inflammatory lung disease, the purified peptide comprising an amino acid sequence of Ac-WGKEQCVE(Nle)(Pego3)-Hdc (SEQ ID NO: 24).

Embodiment 41: The peptide of any one of embodiments 38-40, wherein the inflammatory lung disease comprises asthma, chronic obstructive pulmonary disease (COPD), or COVID-19.

Embodiment 42: The peptide of embodiment 41, wherein asthma is selected from a group consisting of type 2 asthma, non-type 2 asthma, early onset asthma, late-onset asthma, obesity-associated asthma, exercise-induced asthma, asthma exacerbations, asthma associated with pollutants to cigarette smoke, or asthma associated with infections.

Embodiment 43: The peptide of any one of embodiments 38-42, wherein the purified peptide is in a preparation for aerosolization or subcutaneous injection.

Embodiment 44: The peptide of any one of embodiments 38-43, wherein the purified peptide is administered via nasal inhalation, subcutaneously, or orally.

Embodiment 45: The peptide of any one of embodiments 38-44, wherein the method further comprises administering the purified peptide with one or more additional therapeutic agents.

Embodiment 46: The peptide of embodiment 45, wherein the one or more therapeutic agents are administered concurrently or in series with the purified peptide.

Embodiment 47: The peptide of embodiment 45 or embodiment 46, wherein the one or more therapeutic agents comprise short-acting beta2-adrenoceptor agonists (SABA), long-acting beta agonist (LABA), anticholinergic medications, leukotriene receptor antagonists, or a combination thereof.

Embodiment 48: The peptide of embodiment 47, wherein anticholinergic medications comprise ipratropium bromide, inhaled corticosteroids, or systemic corticosteroids.

Embodiment 49: The peptide of embodiment 48, wherein inhaled corticosteroids or systemic corticosteroids comprise budesonide, fluticasone, mometasone, or ciclesonide.

Embodiment 50: A method of treating coronavirus disease 2019 (COVID-19) in a subject in need thereof, the method comprising administering a therapeutically effective amount of a purified peptide comprising an amino acid sequence of KEQCVE (SEQ ID NO: 9) to the subject.

Embodiment 51: The method of embodiment 50, wherein the peptide is selected from a group consisting of SEQ ID NO: 4, SEQ ID NO: 8, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, and SEQ ID NO: 24.

Embodiment 52: A method of treating coronavirus disease 2019 (COVID-19) in a subject in need thereof, the method comprising administering a therapeutically effective amount of a purified peptide comprising an amino acid sequence of Ac-WGKEQCVE(Nle)(Pego3)-Hdc (SEQ ID NO: 24) to the subject.

Embodiment 53: The method of any one of embodiments 50-52, wherein the purified peptide is in a preparation for aerosolization or subcutaneous injection.

Embodiment 54: The method of any one of embodiments 50-53, wherein the purified peptide is administered via nasal inhalation, subcutaneously, or orally.

Embodiment 55: The method of any one of embodiments 50-54, further comprising administering the purified peptide with one or more additional therapeutic agents.

Embodiment 56: The method of embodiment 55, wherein the one or more therapeutic agents are administered concurrently or in series with the purified peptide.

Embodiment 57: The method of embodiment 55 or embodiment 56, wherein the one or more therapeutic agents comprise short-acting beta2-adrenoceptor agonists (SABA), long-acting beta agonist (LABA), anticholinergic medications, leukotriene receptor antagonists, or a combination thereof.

Embodiment 58: The method of embodiment 57, wherein anticholinergic medications comprise ipratropium bromide, inhaled epinephrine, and inhaled corticosteroids.

Embodiment 59: The method of embodiment 58, wherein inhaled corticosteroids comprise budesonide, fluticasone, mometasone, or ciclesonide.

Embodiment 60: A purified peptide for use in a method of treating coronavirus disease 2019 (COVID-19), the purified peptide comprising an amino acid sequence of KEQCVE (SEQ ID NO: 9).

Embodiment 61: The peptide of embodiment 60, wherein the peptide is selected from a group consisting of SEQ ID NO: 4, SEQ ID NO: 8, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, and SEQ ID NO: 24.

Embodiment 62: A purified peptide for use in a method of treating coronavirus disease 2019 (COVID-19), the purified peptide comprising an amino acid sequence of Ac-WGKEQCVE(Nle)(Pego3)-Hdc (SEQ ID NO: 24).

Embodiment 63: The peptide of any one of embodiments 60-62, wherein the purified peptide is in a preparation for aerosolization or subcutaneous injection.

Embodiment 64: The peptide of any one of embodiments 60-62, wherein the purified peptide is administered via nasal inhalation, subcutaneously, or orally.

Embodiment 65: The peptide of any one of embodiments 60-64, wherein the method further comprises administering the purified peptide with one or more additional therapeutic agents.

Embodiment 66: The peptide of embodiment 65, wherein the one or more therapeutic agents are administered concurrently or in series with the purified peptide.

Embodiment 67: The peptide of embodiment 65 or embodiment 66, wherein the one or more therapeutic agents comprise short-acting beta2-adrenoceptor agonists (SABA), long-acting beta agonist (LABA), anticholinergic medications, leukotriene receptor antagonists, or a combination thereof.

Embodiment 68: The peptide of embodiment 67, wherein anticholinergic medications comprise ipratropium bromide, inhaled corticosteroids, or systemic corticosteroids.

Embodiment 69: The peptide of embodiment 68, wherein inhaled corticosteroids or systemic corticosteroids comprise budesonide, fluticasone, mometasone, or ciclesonide.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A composition comprising a surfactant protein A (SP-A) peptide analogue comprising a purified peptide comprising an amino acid sequence according to Ac-WGKEQCVE(Nle)(Pego3)-Hdc (SEQ ID NO: 24).

2. The composition of claim 1, wherein the composition is a pharmaceutical composition.

3. The composition of claim 2, wherein the pharmaceutical composition comprises a pharmaceutically acceptable carrier.

4. The composition of claim 1, wherein the composition is in a preparation for aerosolization, subcutaneous injection, or pulmonary delivery.

5. A system comprising the composition according to claim 1, and a device for pulmonary delivery of said composition.

6. The system of claim 5, wherein said device is a metered dose inhaler or a nebulizer.

7. A method of enhancing SP-A activity in a cell, comprising: delivering the composition according to claim 1 to said cell.

8. The method of claim 7, wherein said cell is a lung cell or a nasal cell

9. The method of claim 7, wherein said cell is in vivo.

10. The method of claim 7, wherein said composition reduces mucin production and/or reduces eosinophilia in said cell.

11. The method of claim 7, wherein said cell is in a subject diagnosed with an inflammatory lung disease; wherein the inflammatory lung disease comprises asthma, chronic obstructive pulmonary disease (COPD), or COVID-19.

12. A method of treating or preventing an inflammatory lung disease in a subject in need thereof, the method comprising administering a therapeutically effective amount of a composition according to claim 1 to the subject.

13. A method of treating or preventing an inflammatory lung disease in a subject in need thereof, the method comprising administering a therapeutically effective amount of a purified peptide comprising an amino acid sequence of Ac-WGKEQCVE(Nle)(Pego3)-Hdc (SEQ ID NO: 24) to the subject.

14. The method of claim 13, wherein the inflammatory lung disease comprises asthma, chronic obstructive pulmonary disease (COPD), or COVID-19.

15. The method of claim 14, wherein asthma is selected from a group consisting of type 2 asthma, non-type 2 asthma, early onset asthma, late onset asthma, obesity associated asthma, exercise induced asthma, asthma exacerbations, asthma associated with pollutants to cigarette smoke, or asthma associated with infections.

16. The method of claim 13, wherein the purified peptide is in a preparation for aerosolization or subcutaneous injection.

17. The method of claim 13, wherein the purified peptide is administered via nasal inhalation, subcutaneously, or orally.

18. The method of claim 13, further comprising administering the purified peptide with one or more additional therapeutic agents.

19. The method of claim 18, wherein the one or more therapeutic agents are administered concurrently or in series with the purified peptide.

20. A purified peptide comprising an amino acid sequence according to Ac-WGKEQCVE(Nle)(Pego3)-Hdc (SEQ ID NO: 24).
